(19) United States
(12) Patent Application Publication
(10) Pub. No.: US 2024/0108632 A1
(43) Pub. Date: Apr. 4, 2024

(54) PHARMACEUTICAL COMPOSITION FOR ADMINISTRATION AS OPHTHALMIC DROP TO PATIENT REQUIRING OPTIC NERVE PROTECTION

(71) Applicants: PINOTBIO, INC., Suwon-si (KR); AJOU UNIVERSITY INDUSTRY-ACADEMIC COOPERATION FOUNDATION, Suwon-si (KR)

(72) Inventors: Young-joon Park, Seoul (KR); Sang-won Jeon, Suwon-si (KR); Ju-yeong Kim, Suwon-si (KR); Jin-soo Lee, Suwon-si (KR); Hyun-yong Cho, Suwon-si (KR)

(73) Assignees: PINOTBIO, INC., Suwon-si (KR); AJOU UNIVERSITY INDUSTRY-ACADEMIC COOPERATION FOUNDATION, Suwon-si (KR)

(21) Appl. No.: 18/141,086

(22) Filed: Apr. 28, 2023

Related U.S. Application Data
(63) Continuation-in-part of application No. PCT/KR21/15441, filed on Oct. 29, 2021.

(30) Foreign Application Priority Data
Oct. 29, 2020 (KR) 10-2020-0142465

(51) Int. Cl. A61K 31/549; A61K 9/00; A61K 47/69; A61P 27/06

(52) U.S. Cl. CPC A61K 31/549; A61K 9/0048; A61K 47/6951; A61P 27/06

(57) ABSTRACT

Ophthalmic therapeutic agents having as pharmaceutically active ingredient a poorly water soluble drug of formula 1 are described. Specifically, an ophthalmic formulation containing an inclusion complex of a poorly soluble drug of formula 1 enclosed in cyclodextrin or a cyclodextrin derivative in an aqueous solution of pH 10 or higher is administered to a patient in need of optic nerve protection.

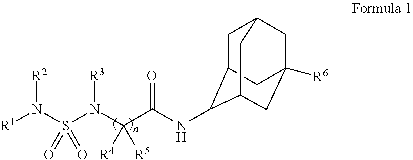

### CROSS REFERENCE TO RELATED APPLICATIONS

This application is a bypass continuation-in-part application of PCT/KR2021/015441 filed Oct. 29, 2021, which claims priority based on Korean Patent Application No. 10-2020-0142465 filed Oct. 29, 2020, of which entire contents are incorporated by reference.

### FIELD

The present invention relates to an ophthalmic treatment having as pharmaceutically active ingredient a poorly soluble drug of Formula 1. Specifically, an ophthalmic formulation comprising an inclusion complex of a poorly water soluble drug of Formula 1 entrapped in cyclodextrin or a cyclodextrin derivative in an aqueous solution at pH 10 or higher for delivery through the corneal epithelium and into the ocular tissues is administered to a patient in need of optic nerve protection.

### BACKGROUND

The eye is surrounded by three types of membranes, the outermost membrane is called the sclera, the innermost membrane through which nerves are distributed is called the retina, and the middle membrane is called the uvea. The uvea is a soft, thin membrane with many blood vessels, and consists of the iris that regulates the amount of light, the ciliary body that supports the lens, and the choroid that blocks light from outside the eye.

The eye maintains intraocular pressure (IOP) through the circulation of aqueous humor. Aqueous humor is a fluid that maintains constant pressure and nutrition within the eye. When the amount of aqueous humor fails to regulate due to aging, stress, or genetics, IOP rises, causing the eyeball to swell and compress the retinal optic nerve, leading to ischemic damage.

Glaucoma is a type of ischemic optic neuropathy in which the optic nerve is damaged, resulting in impaired vision. Elevated intraocular pressure is the main cause, but even with normal intraocular pressure, the optic nerve can be damaged and cause glaucoma. The optic nerve is responsible for transmitting light from the eye to the brain, and when intraocular pressure increases, it presses on the nerve, damaging it and narrowing the vision. Once damaged, the optic nerve cannot be repaired and can lead to blindness if left untreated. Globally, the prevalence in adults is between 0.5 and 4 percent, accounting for about 15 percent of blindness worldwide.

The space between the cornea and the lens in the front part of the eye is filled with a transparent fluid called aqueous humor. The aqueous humor is produced by the ciliary body of the eye. Most of it leaves the eye through the Schlemm's hole at the edge of the iris (a venous system between the iris and cornea), and some through the uvea and sclera. This maintains intraocular pressure and delivers nutrients to the cornea and lens.

Aqueous humor maintains a constant intraocular pressure of 15 to 20 mmHg. However, if there is an increase in the production of aqueous humor or a decrease in its outflow through the trabecular meshwork and the canal of Schlemm, the intraocular pressure can rise, leading to mechanical compression of the optic nerve and potential damage.

Typically, glaucoma medications work by inhibiting the production of aqueous humor or increasing the drainage of aqueous humor to lower intraocular pressure, which prevents damage to the optic nerve and prevents glaucoma from worsening. The most common medications are eye drops, which are applied topically to the eye, although some small amounts may be absorbed systemically. Some oral and injectable medications are available, but they are limited due to the risk of systemic side effects. Glaucoma can worsen even when there are no symptoms, so medication should be continued even if there are no symptoms. Medications that inhibit the production of aqueous humor include carbonic anhydrase inhibitors and beta-blockers. Medications that increase the outflow of aqueous humor include alpha-2 agonists, parasympathomimetics, and prostaglandin agents. Alpha-2 agonists inhibit aqueous production and promote aqueous outflow.

Carbonic anhydrase inhibitors inhibit carbonic anhydrase in the ciliary processes of the eye, which reduces the production of bicarbonate (HCO³⁻), a component of aqueous humor, thereby reducing aqueous humor production and thus lowering IOP. Carbonic anhydrase inhibitors have the side effect of altering electrolyte levels and making the body more acidic.

β (beta) receptors of the sympathetic nervous system are distributed in the blood vessels of the ciliary body, which is responsible for the production of aqueous humor in the eye. When the sympathetic nervous system is activated, the blood vessels of the ciliary body dilate, increasing blood flow and promoting the production of aqueous humor. Beta blockers, specifically timolol, have long been used to reduce the production of aqueous humor and lower intraocular pressure. Betaxolol, another beta blocker, is preferred for patients with lung disease as it has reduced side effects on the lungs. Additionally, betaxolol has a protective effect on the optic nerve in patients with normal intraocular pressure, even in cases of glaucoma. However, local ophthalmic administration of beta blockers can result in systemic absorption, which may lead to systemic side effects.

Alpha-2 agonists act on the alpha-2 receptors of the sympathetic nervous system in the eye, inhibiting the production of aqueous humor and increasing its outflow through the uvea and trabecular meshwork. This leads to a reduction in intraocular pressure. Brimonidine, an alpha-2 agonist, also has a protective effect on the optic nerve. Initially, it can rapidly lower intraocular pressure, but its long-term efficacy in lowering intraocular pressure may diminish over time.

When the parasympathetic nervous system is stimulated, it contracts the ciliary muscles, which adjust the size of the pupil, leading to pupil constriction and increased aqueous humor outflow. Parasympathomimetic agents are used in the diagnosis and treatment of conditions such as glaucoma through their miotic (pupil constriction) effects. Carbachol is administered by injection and should not be used in patients with inflammation of the iris or corneal damage. It may worsen symptoms in patients with conditions such as bronchial asthma, congestive heart failure, hyperthyroidism, gastrointestinal obstruction, urinary obstruction, peptic ulcer, or Parkinson's disease.

Prostaglandin preparations bind to prostaglandin receptors on the ciliary body of the eye, relaxing the ciliary muscles and increasing aqueous humor drainage into the uvea and sclera, thereby reducing intraocular pressure. They have fewer side effects compared to other medications and only require once-daily administration.

Osmotic diuretics reduce intraocular pressure by moving fluid from the vitreous humor towards the blood vessels through osmotic action, leading to a decrease in the volume of the vitreous humor.

The most significant risk factor for glaucoma is elevated intraocular pressure, but in cases of normal-tension glaucoma where intraocular pressure is within the normal range, impaired blood flow has been recognized to play an important role in the development of glaucoma. Systemic conditions that are expected to affect blood supply to the optic nerve include diabetes, hypertension, and hypotension. Hypotension can decrease ocular perfusion pressure and result in reduced blood flow to the optic nerve, particularly during the nocturnal or early morning hours when intraocular pressure tends to rise, making it particularly detrimental.

Recently, there has been significant research on neuroprotective therapies that directly target retinal ganglion cells, which are the cells of the optic nerve. Interest and research in neuroprotective therapy have gained momentum with the understanding that regardless of the underlying mechanisms of glaucoma development, the ultimate common pathway leading to the demise of nerve cells is cellular apoptosis. Various studies are investigating direct treatments targeting retinal ganglion cells to provide neuroprotection.

In recent years, the activity of 11β-hydroxysteroid dehydrogenase type 1 (11β-HSD1) has been reported to be related to trabecular meshwork damage and elevated intraocular pressure. Administration of the non-specific 11β-HSD1 inhibitor carbenoxolone has been shown to reduce intraocular pressure to levels as low as 20% in normal patients. It is known as a drug with superior intraocular pressure-lowering and neuroprotective effects compared to other glaucoma medications.

Most glaucoma medications are formulated as ophthalmic solutions for topical administration in order to achieve therapeutic effects. Products containing commonly used glaucoma treatment agents such as dorzolamide or latanoprost are developed in the form of eye drops, which allow direct application to the treatment area.

On the other hand, insoluble compounds are often dispersed as microparticles and formulated as suspensions to create ophthalmic solutions. In the case of suspensions, in order to sterilize the main ingredient, sterile substances need to be used, and when administered as a suspension, it may exist in a cloudy state on the ocular surface, causing visual disturbances or discomfort during use. This limits the usability of suspension eye drops. To overcome these issues, some formulations utilize oil-based emulsions in which insoluble compounds are dissolved.

A notable example is the use of an oil-based eye drop formulation where a poorly soluble compound such as cyclosporine is solubilized in an oil phase. Oil-based eye drops form a less opaque shape in the eye compared to suspensions, providing an improvement over suspensions in terms of transparency. However, they still have drawbacks such as visual disturbances and discomfort during administration, although to a lesser extent compared to suspension formulations.

To overcome these problems, ophthalmic formulations of poorly water-soluble compounds have been developed in the form of a nanoemulsion-type solution to increase transparency at the time of instillation. These formulations have improved transparency, but because the nanoemulsion is prepared by adding more than 10% surfactant, it is irritating to the ocular mucosa and often causes great discomfort during instillation, which leads to reluctance to use eye drops and reduces treatment effectiveness.

Therefore, there is a need for the development of glaucoma therapeutic formulations that minimize visual disturbances, discomfort, and maintain a stable liquid form during eye drop administration.

On the other hand, due to the lack of ophthalmic therapeutic agents with direct optic nerve protection, the indirect treatment method of reducing ocular load by lowering intraocular pressure is used almost exclusively for the treatment of ischemic optic neuropathy. However, there is an urgent need for therapeutic agents that can inhibit optic nerve damage through direct neuroprotective effects for the continued pharmacological treatment and management of glaucoma.

### SUMMARY

Based on various solubilization techniques and studies on the delivery of a poorly water-soluble drug with Formula 1 into ocular tissues through the corneal epithelium, the inventors have developed an ophthalmic formulation solubilized in the form of a transparent solution. Particularly, when cyclodextrin and its derivatives are used in specific ratios, the poorly soluble drug of Formula 1 can be completely dissolved in therapeutically effective concentrations, maintaining a transparent solution with physical and chemical stability over a certain period of time. It has been discovered that this formulation can penetrate through the corneal epithelium and be delivered into ocular tissues for the treatment of glaucoma. Additionally, a method for manufacturing a transparent ophthalmic formulation has been developed, where cyclodextrin or its derivatives are first dissolved at an alkaline pH, followed by the addition of the poorly soluble drug of Formula 1 and agitation, resulting in the easy production of a transparent solution.

Therefore, the present invention is based on these findings.

The first aspect of the present invention provides an ophthalmic formulation in the form of an eye drop containing an inclusion complex of a poorly water-soluble drug of Formula 1 below entrapped in cyclodextrin or a cyclodextrin derivative in an aqueous solution of pH 10 or higher, allowing it to pass through the corneal epithelium and be delivered into ocular tissues.

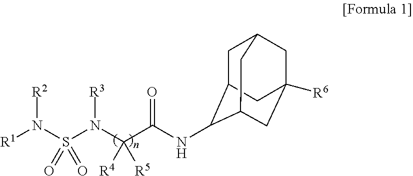

Herein, through the inclusion complex, a sufficient concentration of the poorly soluble drug with Formula 1 can penetrate the corneal epithelium and be delivered into ocular tissues. Moreover, an adequate amount of the poorly soluble drug of Formula 1 can reach ocular tissues through the inclusion complex, providing a degree of protection against ischemic cell damage and preventing cell death.

The second aspect of the present invention provides a pharmaceutical composition containing an inclusion complex, wherein the poorly soluble drug of Formula 1 is entrapped within 2-hydroxypropyl-β-cyclodextrin in an aqueous solution of pH 10 or higher.

The third aspect of the present invention provides a pharmaceutical composition for administration as an ophthalmic formulation in the form of an eye drop to patients in need of optic nerve protection, wherein the pharmaceutical composition contains the poorly soluble drug with Formula 1 to be delivered into ocular tissues by passing through the corneal epithelium.

This pharmaceutical composition may be used for the prevention or treatment of ischemic optic neuropathy, such as glaucoma.

### BRIEF DESCRIPTION OF THE DRAWINGS/FIGURES

The patent or application file contains at least one drawing executed in color. Copies of this patent or patent application publication with color drawing(s) will be provided by the Office upon request and payment of the necessary fee.

FIG. 1 is a conceptual diagram illustrating the mechanism of an ophthalmic formulation containing the poorly soluble drug with Formula 1, which inhibits the enzyme 11β-hydroxysteroid dehydrogenase type 1 (11β-HSD1) associated with the hormone cortisol, thereby suppressing the increase in intraocular pressure, and also activates the antioxidant factor Nrf2/HO-1 to protect the optic nerve.

FIG. 2 is a graph showing the solubility of the main component observed at different pH levels, according to a specific embodiment of the present invention.

FIG. 3 is a graph showing the solubility of the main component at various concentrations of HP-β-CD (hydroxypropyl-ß-cyclodextrin) without pH adjustment (using purified water), according to a specific embodiment of the present invention.

FIG. 4 is a graph showing the solubility of the main component at various concentrations of HP-β-CD with pH adjustment, according to a specific embodiment of the present invention.

FIG. 5 is a photograph comparing the appearance of samples according to a specific embodiment of the present invention.

FIG. 6 is mass transport profiles of NTX-101_vC (DP using HP-beta CD as solubilizer) or NTX-101_VT (DP using D-α-Tocopherol polyethlylene glycol 1000 succinate (TPGS) as solubilizer) ophthalmic solution in rabbit corneas (n=3). Average drug mass (ng) transported across rabbit cornea membrane up to 3 h at the vertical type or side-by-side type diffusion experiment set up. Mean ±SE (standard error) (n=3).

FIGS. 7A-7G. are pharmacokinetic profiles in plasma or ocular tissues after ophthalmic administration (100 μL of NTX-101_vC or NTX-101_VT into each eye in New Zealand White rabbits (n=3). Average drug concentration in (A) plasma, (B) aqueous humor, (C) vitreous humor, (D) cornea, (E) conjunctiva, (F) iris/ciliary body, and (G) retina of individual treatment group rabbit (ng/g of tissues; ng/ml in case of plasma or humors) up to 24 hr after drug administration. Mean ±SE (standard error) (n=3).

FIGS. 8A-8D are pharmacokinetic parameters (AUC or Cmax) in ocular tissues normalized by the parameters in plasma in the treatment group (100 μL administration of NTX-101_vC or NTX-101_VT into each eye in New Zealand White rabbits (n=3)). Average ratios of (A) AUC_24 h in ocular tissues over AUC_24 h in plasma and (B) Cmax in ocular tissues over Cmax in plasma (Cmax, T/Cmax,P) after NTX-101_vC administration. Average ratios of (C) AUC_24 h in ocular tissues over AUC_24 h in plasma and (D) Cmax in ocular tissues over Cmax in plasma (Cmax, T/Cmax,P) after NTX-101_VT administration. Mean ±SE (standard error) (n=3).

FIG. 9. is Scheme of the effect of KR-67607 on glaucoma in DBA/2 mice.

FIG. 10 is intraocular pressure (IOP) of the mouse (4 weeks). IOP of mouse eyes were measured by a rebound tonometer. The mean value of IOP was estimated as an average from six correct single values. *** p<0.001 compared with normal group. ##p<0.01 compared with vehicle group.

FIG. 11 is intraocular pressure (IOP) of the mouse (12 weeks). IOP of mouse eyes were measured by a rebound tonometer. The mean value of IOP was estimated as an average from six correct single values. *** p<0.001 compared with normal group. ###p<0.001 compared with vehicle group.

FIG. 12 is intraocular pressure (IOP) of the mouse (16 weeks). IOP of mouse eyes were measured by a rebound tonometer. The mean value of IOP was estimated as an average from six correct single values. *** p<0.001 compared with normal group. ###p<0.001 compared with vehicle group.

FIG. 13 is intraocular pressure (IOP) of the mouse over a 16-week period. Graph of change in IOP in each experimental group over time. The mean value of IOP was estimated as an average from six correct single values.

FIG. 14 is effect of KR-67607 on optic nerve head damage in DBA/2 mouse. The enucleated eyeball was fixed for preparation of paraffin block. The tissue slide was stained by H&E stain. Optic nerve head of retina was observed using Nanozoomer. The scale bar means 200 um.

FIG. 15 is effect of KR-67607 on optic nerve head damage in DBA/2 mouse. The enucleated eyeball was fixed for preparation of paraffin block. Immunohistochemistry was performed using the anti-cleaved caspase-3 antibody. Optic nerve head of retina was observed using Nanozoomer. The scale bar means 200 μm.

FIG. 16 is effect of KR-67607 on optic nerve head damage in DBA/2 mouse. The enucleated eyeball was fixed for preparation of paraffin block. Immunohistochemistry was performed using the anti-cleaved caspase-9 antibody. Optic nerve head of retina was observed using Nanozoomer. The scale bar means 200 μm.

FIG. 17 is effect of KR-67607 on optic nerve head damage in DBA/2. Cell death in damaged tissue was analyzed by TUNEL assay. The immuno-reactivity was observed by fluorescence microscope. The arrows indicate TUNEL-positive cells. Scale bar means 200 μm. GCL, ganglion cell layer; INL, inner nuclear layer; ONL, outer nuclear layer.

FIG. 18 is effect of KR-67607 on ganglion cell loss in DBA/2 mouse. The enucleated eyeball was fixed for preparation of paraffin block. The tissue slide was stained by H&E stain. The retina was observed using Nanozoomer. The arrows indicate the loss of RGCs. The scale bar means 100 μm. GCL, ganglion cell layer; IPL, inner plexiform layer; INL, inner nuclear layer; OPL, outer plexiform layer; ONL, outer nuclear layer; OS, outer segment; RPE, retinal pigment epithelia.

FIG. 19 is effect of KR-67607 on ganglion cell death in DBA/2 mouse. The enucleated eyeball was fixed for preparation of paraffin block. Immunohistochemistry was performed using the anti-cleaved caspase-3 antibody. The retina was observed using Nanozoomer. The scale bar means 100 μm. GCL, ganglion cell layer; IPL, inner plexiform layer; INL, inner nuclear layer; OPL, outer plexiform layer; ONL, outer nuclear layer; OS, outer segment; RPE, retinal pigment epithelia.

FIG. 20 is effect of KR-67607 on ganglion cell death in DBA/2 mouse. The enucleated eyeball was fixed for preparation of paraffin block. Immunohistochemistry was performed using the anti-cleaved caspase-9 antibody. The retina was observed using Nanozoomer. The scale bar means 100 μm. GCL, ganglion cell layer; IPL, inner plexiform layer; INL, inner nuclear layer; OPL, outer plexiform layer; ONL, outer nuclear layer; OS, outer segment; RPE, retinal pigment epithelia.

FIG. 21 is effect of KR-67607 on ganglion cell loss in DBA/2 mouse. The enucleated eyeball was fixed for preparation of flat mount. The tissue was stained with Brn3a primary antibody. The retina was observed using Confocal. The scale bar means 50 μm.

FIG. 22 is effect of KR-67607 on activation of astrocytes in DBA/2 mouse. The enucleated eyeball was fixed for preparation of flat mount. The tissue was stained with GFAP primary antibody. The retina was observed using Confocal. The scale bar means 100 μm.

FIG. 23 is effect of KR-67607 on change of retinal neuron, cone photoreceptor and cone bipolar cell in DBA/2 mouse. The enucleated eyeball was fixed for preparation of paraffin block. The tissue slide was immuno-stained with GLT-1 primary antibody. The retina was observed using Nanozoomer. The scale bar means 50 μm. GCL, ganglion cell layer; IPL, inner plexiform layer; INL, inner nuclear layer; OPL, outer plexiform layer; ONL, outer nuclear layer.

FIG. 24 is effect of KR-67607 on change of GABAergic amacrine cell in DBA/2 mouse. The enucleated eyeball was fixed for preparation of paraffin block. The tissue slide was immuno-stained with GABA primary antibody. The retina was observed using Nanozoomer. The arrows indicate GABA-positive cells. The scale bar means 50 . m. GCL, ganglion cell layer; IPL, inner plexiform layer; INL, inner nuclear layer; OPL, outer plexiform layer; ONL, outer nuclear layer.

FIG. 25 is effect of KR-67607 on change of Glycinergic amacrine cell in DBA/2 mouse. The enucleated eyeball was fixed for preparation of paraffin block. The tissue slide was immuno-stained with glycine primary antibody. The retina was observed using Nanozoomer. The scale bar means 50 μm. GCL, ganglion cell layer; IPL, inner plexiform layer; INL, inner nuclear layer; OPL, outer plexiform layer; ONL, outer nuclear layer.

FIG. 26 is effect of KR-67607 on change of microglial cell in DBA/2 mouse. The enucleated eyeball was fixed for preparation of paraffin block. The tissue slide was immuno-stained with Iba-1 primary antibody. The retina was observed using Nanozoomer. The arrows indicate Iba-1-positive cells. The scale bar means 50 μm. GCL, ganglion cell layer; IPL, inner plexiform layer; INL, inner nuclear layer; OPL, outer plexiform layer; ONL, outer nuclear layer.

FIG. 27 is a comparative analysis of corneal permeability and intraocular distribution after ophthalmic administration was conducted in rabbit corneas (plotting the permeation rate as a function of time).

FIG. 28 is comparison of mRNA expression of Glial Fibrillary Acidic Protein (GFAP), an indicator of glaucoma severity that increases with elevated intraocular pressure, Tumor Necrosis Factor alpha (TNFα), an inflammatory response factor, and BCL-2associated x protein (BAX), a key factor involved in cellular apoptosis.

FIG. 29 is change in IOP over an eight-hour time course following treatment. The change in IOP of contralateral, control eyes and glaucomatous eyes. IOP change was calculated by [IOP post-treatment-Pre]. Data expressed as the mean ±S.E.M. of 6 eyes.

FIG. 30 is effects of KR-67607 (1.5 or 3.0 mg/mL) on change in IOP (Extra dosing). IOP change was calculated by [IOP post-treatment-Pre]. Data expressed as the mean ±S.E.M. of 3 eyes.

FIG. 31 is effects of test compounds on retinal nerve fiber layer (RNFL) thickness (Total average). RNFL thickness was measured with OCT. Dashed lines indicate laser photocoagulation treatments. Data expressed as the mean ±S.E.M. of 4-6 eyes. * p<0.05; ** p<0.01; *** p<0.001 compared to normal control eye using two-way repeated measures ANOVA followed by Student's t-test.

FIG. 32. is effects of brimonidine and KR-67607 on RNFL thickness (Total average). RNFL thickness was measure with OCT. Dashed lines indicate laser photocoagulation treatments. Data expressed as the mean ±S.E.M. of 4-6 eyes. (Comparing RFNL thickness at 5 weeks, RNFL thickness with KR-67607 treatment was greater than that with vehicle treatment (unpaired t-test, p<0.05)).

### DETAILED DESCRIPTION

Typically, poorly soluble drugs exhibit slow release rates, resulting in delayed absorption and lower bioavailability and expression of efficacy.

The present invention is characterized by the development of a method of solubilizing a poorly water-soluble drug of Formula 1 for delivery through the corneal epithelium and into the ocular tissues, and providing it in an eye drop formulation. In other words, the present invention is designed to increase the bioavailability of a poorly water-soluble drug of Formula 1 through the formation of soluble complexes, using cyclodextrins or derivatives thereof that have an entrapment capacity for hydrophobic molecules, resulting in a formulation, such as an eye drop formulation, with excellent release of the poorly water-soluble drug of Formula 1.

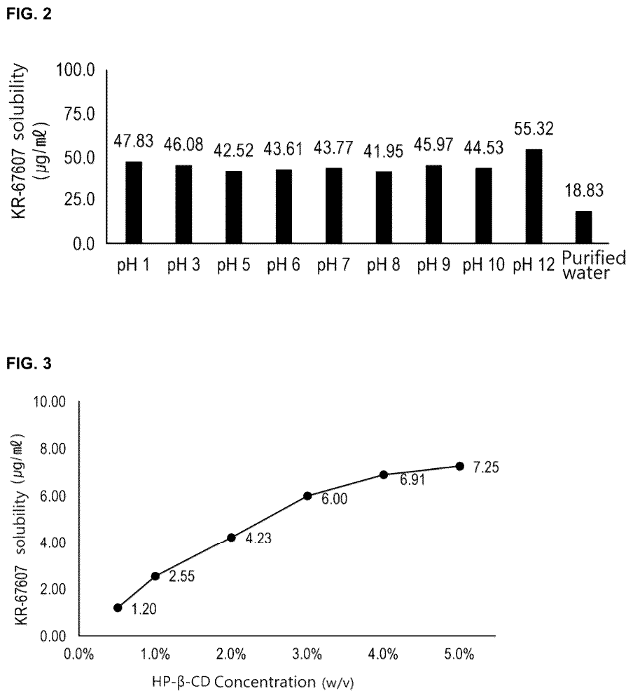

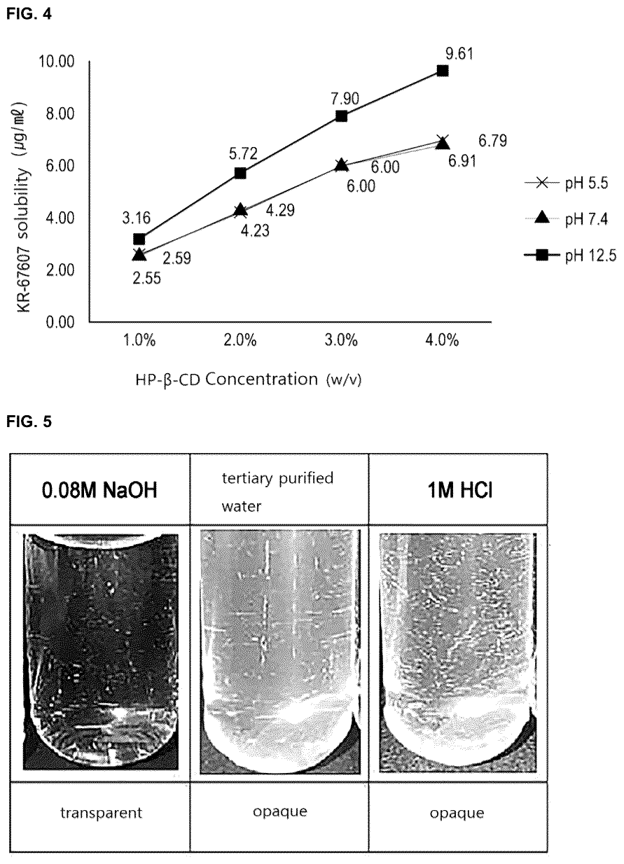

Through experiments, the present invention has found that both purified water and pH 1 to 12 solutions show little difference in solubility with pH for the poorly soluble drug of Formula 1 (FIG. 2 and Table 2), whereas the solubility of the poorly soluble drug of Formula 1 in water increases with increasing concentration of the cyclodextrin derivatives (FIG. 3 and Table 3), and further found that for the poorly soluble drug of Formula 1, the solubility of the cyclodextrin derivatives is basic >acidic, neutral (FIG. 4 and Table 4). FIG. 5 also shows that the entrapment ability of the cyclodextrin derivatives for the poorly soluble drug of Formula 1 is enhanced when the solution prior to entrapment is under alkaline conditions.

When cyclodextrin or its derivatives are used as solubilizers for Formula 1, the resulting ophthalmic formulation not only improves wetting properties due to the enhanced solubility of cyclodextrin or its derivatives but also exhibits significantly increased solubility in water through the amorphization of the inclusion complex. Moreover, the inclusion complex formulation demonstrates much faster drug release compared to physical mixtures or powders.

Therefore, the present invention, from the results of confirming the pharmacological efficacy in terms of lowering intraocular pressure and optic nerve protection through monkey animal experiments, suggests that the poorly soluble drug of Formula 1 can pass through the corneal epithelium through an eye drop formulation and be delivered into the ocular tissue at a sufficient concentration (Examples 31 to 35).

In addition, the present invention has found that the eye drop formulation of the poorly soluble compound of Formula 1 according to the present invention not only exerts a strong optic neuroprotective effect by inhibiting the enzyme activity of 11β-HSD1 distributed in the retina and optic nerve in the eye through pharmacological mechanism and efficacy at the cellular level, but also reaches the ocular tissue in an amount sufficient to prevent cell death caused by ischemic damage(Examples 31-35).

The present invention is based on these findings.

### [Poorly Soluble Drug of Formula 1]

In the present specification, the poorly soluble drug of Formula 1 may be a compound represented by Formula 1 below or a pharmaceutically acceptable salt thereof.

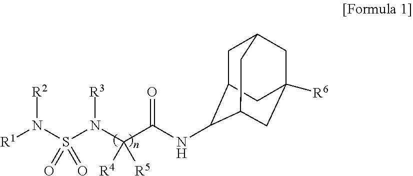

Herein, R¹ may be H; C₁-C₆ alkyl; cyano C₁-C₆ alkyl, C₃-C₈ cycloalkyl; benzyl, unsubstituted or substituted with halogen, C₁-C₆ alkyl or OCX₃ (X is halogen); phenylethyl; C₁-C₆ alkoxycarbonyl; phenylacetyl; naphthyl; or halogen, C₁-C₆ alkyl, C₃-C₈ cycloalkyl, C₁-C₆ alkoxy, CX₃ (X is halogen), OCX₃ (X is halogen) cyano, nitro, or 5- to 10-membered aryl substituted with aryl or heteroaryl.

Herein, R² and R³ may each independently be C₁-C₆ alkyl; C₂-C₆ alkenyl; or a cyclic structure in which R² and R³ form a ring.

Herein, the ring structure which R² and R³ bind together to form may be

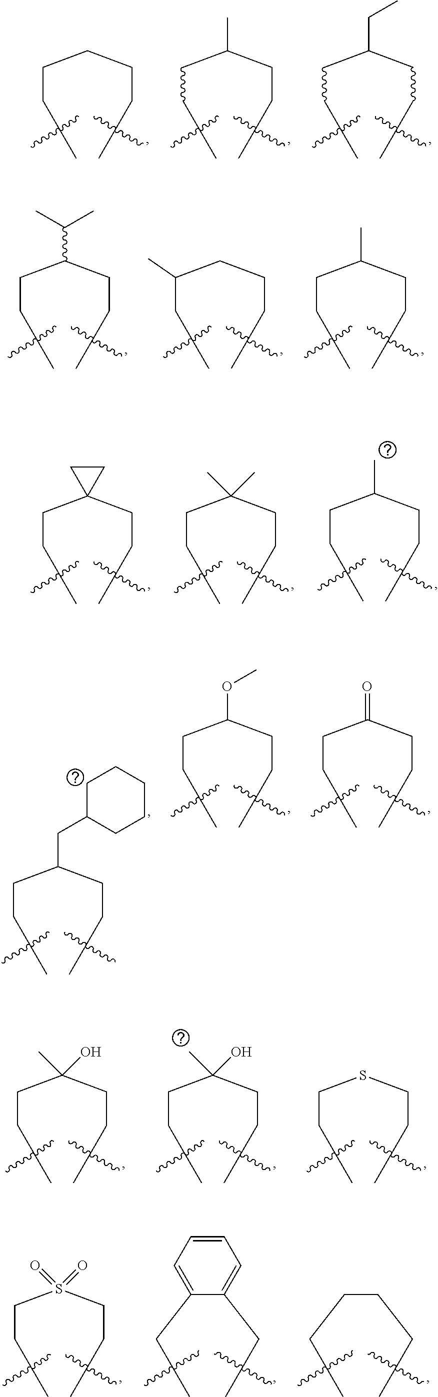

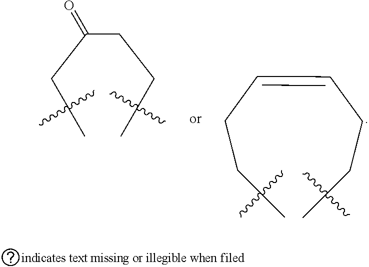 [SMILES: *CCC(=O)C* ; *CC/C=C\CC*]

Herein, R⁴ and R⁵ independently may be H; or C₁-C₆ alkyl; R⁶ may be H; OH; COOR⁷; or CONR⁷R⁷; R⁷ may be H; or C₁-C₆ alkyl; and n may be an integer of 1-3.

The term used herein “alkyl” means a linear or branched, saturated hydrocarbon group and includes, for example, methyl, ethyl, propyl, isobutyl, pentyl and hexyl. C₁-C₆ alkyl means an alkyl group having an alkyl unit of 1 to 6 carbons, excluding the number of carbons of a substituent when the C₁-C₆ alkyl is substituted. In Formula 1, C₁-C₆ alkyl at R¹ is preferably C₁-C₄ alkyl, more preferably C₁-C₂ alkyl.

As used herein, the term “halogen” refers to a halogen element and includes, for example, fluoro, chloro, bromo and iodo.

The term “alkenyl” refers to linear or branched, unsaturated hydrocarbon group having given number of carbons and includes, for example, ethenyl, vinyl, propenyl, allyl, isopropenyl, butenyl, isobutenyl, t-butenyl, n-pentenyl and n-hexenyl. In Formula 1, C₂-C₆ alkenyl at R² or R³ means an alkenyl group having an alkenyl unit of 2 to 6 carbons, excluding the number of carbons of a substituent when the C₂-C₆ alkenyl is substituted.

The term “aryl” refers to a substituted or unsubstituted, monocyclic or polycyclic carbon ring which is entirely or partially unsaturated.

The term “heteroaryl” refers to a heterocyclic aromatic group which contains oxygen, sulfur or nitrogen, preferably oxygen as a heteroatom. The number of the heteroatom is 1-4, preferably 1-2. In the heteroaryl, the aryl may be specifically monoaryl or biaryl.

The term used herein “alkoxy” means a radical formed by removing hydrogen from alcohol. Where C₁-C₆ alkoxy is substituted, the number of carbons of a substituent is excluded.

According to an embodiment of the present invention, R¹ in Formula 1 represents phenyl or naphthalene group substitutued with halogen, C₁-C₆ alkyl, C₃-C₈ cycloalkyl, C₁-C₆ alkoxy, CF₃, OCF₃, cyano, nitro, or 5-10 membered aryl or heteroaryl; n represents an integer of 1.

According to another embodiment of the present invention, the compound represented by the Formula 1 may be one selected from the group consisting of:
(1) N-(adamantan-2-yl)-2-(1,1-dioxido-6-(2-oxo-2-phenylethyl)-1,2,6-thiadiazinan-2-yl)acetamide;
(2) N-(adamantan-2-yl)-2-(1,1-dioxido-6-phenyl-1,2,6-thiadiazinan-2-yl)acetamide;
(3) tert-butyl 6-(2-(adamantan-2-ylamino)-2-oxoethyl)-1,2,6-thiadiazinan-2-carboxylate-1,1-dioxide;
(4) N-(adamantan-2-yl)-2-(1,1-dioxido-1,2,6-thiadiazinan-2-yl)acetamide hydro chloride;
(5) N-(adamantan-2-yl)-2-(6-benzyl-1,1-dioxido-1,2,6-thiadiazinan-2-yl)acetamide;
(6) N-(adamantan-2-yl)-2-(6-(4-fluorobenzyl)-1, 1-dioxido-1,2,6-thiadiazinan-2-yl)acetamide;
(7) N-(adamantan-2-yl)-2-(1,1-dioxido-6-(4-(trifluoromethoxy)benzyl)-1,2,6-thiadiazinan-2-yl)acetamide;
(8) N-(adamantan-2-yl)-2-(6-(4-chlorobenzyl)-1, 1-dioxido-1,2,6-thiadiazinan-2-yl)acetamide;
(9) N-(adamantan-2-yl)-2-(6-(3-methylbenzyl)-1,1-dioxido-1,2,6-thiadiazinan-2-yl)acetamide;
(10) N-(adamantan-2-yl)-2-(6-(3-chlorobenzyl)-1, 1-dioxido-1,2,6-thiadiazinan-2-yl)acetamide;
(11) N-(adamantan-2-yl)-2-(6-(3-fluorobenzyl)-1,1-dioxido-1,2,6-thiadiazinan-2-yl)acetamide;
(12) N-(adamantan-2-yl)-2-(6-(3-methoxybenzyl)-1, 1-dioxido-1,2,6-thiadiazinan-2-yl)acetamide;
(13) N-(adamantan-2-yl)-2-(6-(2-chlorobenzyl)-1, 1-dioxido-1,2,6-thiadiazinan-2-yl)acetamide;
(14) N-(adamantan-2-yl)-2-(6-(2-fluorobenzyl)-1,1-dioxido-1,2,6-thiadiazinan-2-yl)acetamide;
(15) N-(adamantan-2-yl)-2-(1,1-dioxido-6-phenethyl-1,2,6-thiadiazinan-2-yl)acetamide;
(16) N-(adamantan-2-yl)-2-(6-(3-fluorophenyl)-1, 1-dioxido-1,2,6-thiadiazinan-2-yl)acetamide;
(17) N-(adamantan-2-yl)-2-(6-(3-chlorophenyl)-1, 1-dioxido-1,2,6-thiadiazinan-2-yl)acetamide;
(18) N-(adamantan-2-yl)-2-(6-(4-fluorophenyl)-1, 1-dioxido-1,2,6-thiadiazinan-2-yl)acetamide;
(19) N-(adamantan-2-yl)-2-(6-(4-chlorophenyl)-1, 1-dioxido-1,2,6-thiadiazinan-2-yl)acetamide;
(20) N-(adamantan-2-yl)-2-(6-(4-methoxyphenyl)-1, 1-dioxido-1,2,6-thiadiazinan-2-yl)acetamide;
(21) N-(adamantan-2-yl)-2-(6-(3-methoxyphenyl)-1,1-dioxido-1,2,6-thiadiazinan-2-yl)acetamide;
(22) N-(adamantan-2-yl)-2-(6-(2-fluorophenyl)-1, 1-dioxido-1,2,6-thiadiazinan-2-yl)acetamide;
(23) N-(adamantan-2-yl)-2-(6-(3,4-dichlorophenyl)-1, 1-dioxido-1,2,6-thiadiazinan-2-yl)acetamide;
(24) N-(adamantan-2-yl)-2-(1,1-dioxido-6-(p-tolyl)-1,2,6-thiadiazinan-2-yl)acetamide;
(25) N-(adamantan-2-yl)-2-(1,1-dioxido-5-phenyl-1,2,5-thiadiazolidin-2-yl)acetamide;
(26) N-(adamantan-2-yl)-2-(6-methyl-1, 1-dioxido-1,2,6-thiadiazinan-2-yl)acetamide;
(27) N-(adamantan-2-yl)-2-(6-(4-chlorophenyl)-1,1-dioxido-1,2,6-thiadiazinan-2-yl)acetamide;
(28) Methyl-4-(2-(6-(4-chlorophenyl)-1, 1-dioxido-1,2,6-thiadiazinan-2-yl) acetamido)adamantan-1-carboxylate;
(29) 4-(2-(6-(4-chlorophenyl)-1, 1-dioxido-1,2,6-thiadiazinan-2-yl)acetamido) adamantan-1-carboxylic acid;
(30) N-(adamantan-2-yl)-2-(1,1-dioxido-6-(4-(trifluoromethyl)phenyl)-1,2,6-thiadiazinan-2-yl)acetamide;
(31) N-(adamantan-2-yl)-2-(6-(4-cyanophenyl)-1, 1-dioxido-1,2,6-thiadiazinan-2-yl)acetamide;
(32) N-(adamantan-2-yl)-2-(6-(naphthalene-2-yl)-1, 1-dioxido-1,2,6-thiadiazinan-2-yl)acetamide;
(33) Methyl-4-(2-(1,1-dioxido-6-phenyl-1,2,6-thiadiazinan-2-yl)acetamido)adamantan-1-carboxylate;
(34) 4-(2-(6-(4-chlorophenyl)-1, 1-dioxido-1,2,6-thiadiazinan-2-yl)acetamido)adamantan-1-carboxamide;
(35) N-(adamantan-2-yl)-2-(6-cyclohexyl-1, 1-dioxido-1,2,6-thiadiazinan-2-yl)acetamide;
(36) 4-(2-(1,1-dioxido-6-phenyl-1,2,6-thiadiazinan-2-yl)acetamido)adamantan-1-carboxylic acid;
(37) 2-(6-(4-chlorophenyl)-1, 1-dioxido-1,2,6-thiadiazinan-2-yl)-N-(5-hydroxyadamantan-2-yl)acetamide;
(38) 2-(1,1-dioxido-6-phenyl-1,2,6-thiadiazinan-2-yl)-N-(5-hydroxyadamantan-2-yl)acetamide;
(39) 2-(1,1-dioxido-6-phenyl-1,2,6-thiadiazinan-2-yl)-N-(5-hydroxyadamantan-2-yl)acetamide;
(40) 2-(6-(4-fluorophenyl)-1, 1-dioxido-1,2,6-thiadiazinan-2-yl)-N-(5-hydroxyadamantan-2-yl)acetamide;
(41) 2-(6-(2-fluorophenyl)-1, 1-dioxido-1,2,6-thiadiazinan-2-yl)-N-(5-hydroxyadamantan-2-yl)acetamide;
(42) 4-(2-(6-(2-fluorophenyl)-1, 1-dioxido-1,2,6-thiadiazinan-2-yl)acetamido)adamantan-1-carboxamide;
(43) 4-(2-(6-(2-fluorophenyl)-1, 1-dioxido-1,2,6-thiadiazinan-2-yl)acetamido)adamantan-1-carboxamide;
(44) 4-(2-(6-(4-fluorophenyl)-1, 1-dioxido-1,2,6-thiadiazinan-2-yl)acetamido)adamantan-1-carboxamide;
(45) 4-(2-(6-(4-fluorophenyl)-1, 1-dioxido-1,2,6-thiadiazinan-2-yl)acetamido)adamantan-1-carboxamide;
(46) 2-(6-(3,4-dichlorophenyl)-1, 1-dioxido-1,2,6-thiadiazinan-2-yl)-N-(5-hydroxyadamantan-2-yl)acetamide;
(47) 2-(6-(3-chlorophenyl)-1, 1-dioxido-1,2,6-thiadiazinan-2-yl)-N-(5-hydroxyadamantan-2-yl)acetamide;
(48) N-(adamantan-2-yl)-2-(6-ethyl-1, 1-dioxido-1,2,6-thiadiazinan-2-yl)acetamide;
(49) 4-(2-(6-(3,4-dichlorophenyl)-1, 1-dioxido-1,2,6-thiadiazinan-2-yl)-acetamido)adamantan-1-carboxamide;
(50) 4-(2-(6-(3,4-dichlorophenyl)-1,1-dioxido-1,2,6-thiadiazinan-2-yl)-acetamido)adamantan-1-carboxamide;
(51) N-(adamantan-2-yl)-2-(1,1-dioxido-6-(prop-2-yn-1-yl)-1,2,6-thiadiazinan-2-yl)acetamide;
(52) 4-(2-(6-(3-methoxyphenyl)-1, 1-dioxido-1,2,6-thiadiazinan-2-yl)-acetamido)adamantan-1-carboxamide;
(53) 4-(2-(6-(3-methoxyphenyl)-1, 1-dioxido-1,2,6-thiadiazinan-2-yl)-acetamido)adamantan-1-carboxamide;
(54) 4-(2-(1,1-dioxido-6-(p-tolyl)-1,2,6-thiadiazinan-2-yl)acetamido)adamantan-1-carboxamide;
(55) 4-(2-(1,1-dioxido-6-(p-tolyl)-1,2,6-thiadiazinan-2-yl)acetamido)adamantan-1-carboxamide;
(56) 4-(2-(6-(3-chlorophenyl)-1,1-dioxido-1,2,6-thiadiazinan-2-yl)-acetamido)adamantan-1-carboxamide;
(57) N-(5-hydroxyadamantan-2-yl)-2-(6-(napthalene-2-yl)-1, 1-dioxido-1,2,6-thiadiazinan-2-yl)acetamide;
(58) 2-(6-(4-cyanophenyl)-1, 1-dioxido-1,2,6-thiadiazinan-2-yl)-N-(5-hydroxyadamantan-2-yl)acetamide;
(59) 4-(2-(6-(2-fluorophenyl)-1, 1-dioxido-1,2,6-thiadiazinan-2-yl)-acetamido)adamantan-1-carboxamide;
(60) 4-(2-(1,1-dioxido-6-phenyl-1,2,6-thiadiazinan-2-yl)acetamido)adamantan-1-carboxamide;
(61) 4-(2-(1,1-dioxido-5-phenyl-1,2,5-thiadiazolidin-2-yl)acetamido)adamantan-1-carboxamide;
(62) 4-(2-(1,1-dioxido-6-(4-trifluoromethyl)phenyl-1,2,6-thiadiazinan-2-yl)acetamido)adamantan-1-carboxamide;
(63) 4-(2-(6-(napthalene-2-yl)-1, 1-dioxido-1,2,6-thiadiazinan-2-yl)acetamido)adamantan-1-carboxamide;
(64) 4-(2-(5-(2-fluorophenyl)-1, 1-dioxido-1,2,5-thiadiazolidin-2-yl)acetamido)adamantan-1-carboxamide;
(65) 4-(2-(5-(2-chlorophenyl)-1, 1-dioxido-1,2,5-thiadiazolidin-2-yl)acetamido)adamantan-1-carboxamide;
(66) 4-(2-(5-(4-fluorophenyl)-1, 1-dioxido-1,2,5-thiadiazolidin-2-yl)acetamido)adamantan-1-carboxamide;
(67) N-(adamantan-2-yl)-2-(5-(2-chlorophenyl)-1, 1-dioxido-1,2,5-thiadiazolidin-2-yl)acetamide;
(68) N-(adamantan-2-yl)-2-(5-(4-fluorophenyl)-1, 1-dioxido-1,2,5-thiadiazolidin-2-yl)acetamide;
(69) 4-(2-(6-(3-fluorophenyl)-1,1-dioxido-1,2,6-thiadiazinan-2-yl)acetamido)adamantan-1-carboxamide;
(70) 4-(2-(6-(4-methoxyphenyl)-1, 1-dioxido-1,2,6-thiadiazinan-2-yl)acetamido)adamantan-1-carboxamide;
(71) 4-(2-(6-(4-cyanophenyl)-1,1-dioxido-1,2,6-thiadiazinan-2-yl)acetamido)adamantan-1-carboxamide;
(72) 4-(2-(6-(4-chlorophenyl)-1, 1-dioxido-1,2,6-thiadiazinan-2-yl)propaneamido)adamantan-1-carboxamide;
(73) 4-(2-(6-(4-chloronaphtalene-1-yl)-1, 1-dioxido-1,2,6-thiadiazinan-2-yl)acetamido)adamantan-1-carboxamide;
(74) 4-(2-(6-(3,4-dichlorophenyl)-1, 1-dioxido-1,2,6-thiadiazinan-2-yl)propaneamido)adamantan-1-carboxamide;
(75) 4-(2-(6-(2,4-difluorophenyl)-1,1-dioxido-1,2,6-thiadiazinan-2-yl)acetamido)adamantan-1-carboxamide;
(76) 4-(2-(6-(3,4-difluorophenyl)-1, 1-dioxido-1,2,6-thiadiazinan-2-yl)acetamido)adamantan-1-carboxamide;
(77) 4-(2-(1,1-dioxido-5-(o-tolyl)-1,2,5-thiadiazolidin-2-yl)acetamido)adamantan-1-carboxamide;
(78) 4-(2-(5-(benzo[d][1,3]dioxol-5-yl)-1, 1-dioxido-1,2,5-thiadiazolidin-2-yl)acetamido)adamantan-1-carboxamide;
(79) 4-(2-(6-(3,4-difluorophenyl)-1,1-dioxido-1,2,6-thiadiazinan-2-yl)propanemido)adamantan-1-carboxamide;
(80) 4-(2-(6-(2,4-difluorophenyl)-1, 1-dioxido-1,2,6-thiadiazinan-2-yl)propaneamido)adamantan-1-carboxamide;
(81) 4-(2-(6-(benzo[d][1,3]dioxol-5-yl)-1, 1-dioxido-1,2,6-thiadiazinan-2-yl)acetamido)adamantan-1-carboxamide;
(82) 4-(2-(6-(benzo[d][1,3]dioxol-5-yl)-1, 1-dioxido-1,2,6-thiadiazinan-2-yl)propaneamido)adamantan-1-carboxamide;
(83) 4-(2-(6-(2-fluorophenyl)-1, 1-dioxido-1,2,6-thiadiazinan-2-yl)acetamido)adamantan-1-carboxamide;
(84) 4-(2-(6-(2-fluorophenyl)-1, 1-dioxido-1,2,6-thiadiazinan-2-yl)acetamido)adamantan-1-carboxamide;
(85) 4-(2-(6-(2,5-difluorophenyl)-1,1-dioxido-1,2,6-thiadiazinan-2-yl)acetamido) adamantan-1-carboxamide;
(86) 4-(2-(6-(2,5-difluorophenyl)-1,1-dioxido-1,2,6-thiadiazinan-2-yl)acetamido) adamantan-1-carboxamide;
(87) 4-(2-(1,1-dioxido-6-(2,4,6-trichlorophenyl)-1,2,6-thiadiazinan-2-yl)acetamido)adamantan-1-carboxamide;
(88) 4-(2-(1,1-dioxido-6-(2,4,6-trichlorophenyl)-1,2,6-thiadiazinan-2-yl)acetamido)adamantan-1-carboxamide;
(89) 4-(2-(1,1-dioxido-6-(2,4,6-trifluorophenyl)-1,2,6-thiadiazinan-2-yl)acetamido)adamantan-1-carboxamide;
(90) 4-(2-(1,1-dioxido-6-(2,4,6-trifluorophenyl)-1,2,6-thiadiazinan-2-yl)acetamido)adamantan-1-carboxamide;
(91) 4-(2-(6-(2-chlorophenyl)-1,1-dioxido-1,2,6-thiadiazinan-2-yl)acetamido)adamantan-1-carboxamide;
(92) 4-(2-(6-(2-chlorophenyl)-1, 1-dioxido-1,2,6-thiadiazinan-2-yl)acetamido)adamantan-1-carboxamide;
(93) 4-(2-(6-(2-fluorophenyl)-1, 1-dioxido-1,2,6-thiadiazinan-2-yl)propaneamido)adamantan-1-carboxamide;
(94) 4-(2-(6-(2-fluorophenyl)-1, 1-dioxido-1,2,6-thiadiazinan-2-yl)propaneamido)adamantan-1-carboxamide;
(95) 4-(2-(6-(2-bromophenyl)-1, 1-dioxido-1,2,6-thiadiazinan-2-yl)acetamido)adamantan-1-carboxamide;
(96) 4-(2-(1,1-dioxido-6-(2,4,5-trifluorophenyl)-1,2,6-thiadiazinan-2-yl)acetamido)adamantan-1-carboxamide;
(97) 4-(2-(6-(2-fluorophenyl)-1, 1-dioxido-1,2,6-thiadiazinan-2-yl)methylpropaneamido)adamantan-1-carboxamide;
(98) 4-(2-(6-(2-fluorophenyl)-1, 1-dioxido-1,2,6-thiadiazinan-2-yl)methylpropaneamido)adamantan-1-carboxamide;
(99) 4-(2-(6-(2,5-dichlorophenyl)-1, 1-dioxido-1,2,6-thiadiazinan-2-yl)acetamido)adamantan-1-carboxamide;
(100) 4-(2-(1,1-dioxido-6-(2,4,5-trichlorophenyl)-1,2,6-thiadiazinan-2-yl)acetamido)adamantan-1-carboxamide;
(101) 4-(2-(1,1-dioxido-6-(2,4,5-trichlorophenyl)-1,2,6-thiadiazinan-2-yl)acetamido)adamantan-1-carboxamide;
(102) 4-(2-(6-(2,6-difluorophenyl)-1,1-dioxido-1,2,6-thiadiazinan-2-yl)acetamido)adamantan-1-carboxamide;
(103) 4-(2-(6-(2,6-difluorophenyl)-1, 1-dioxido-1,2,6-thiadiazinan-2-yl)acetamido)adamantan-1-carboxamide;
(104) 4-(2-(6-(2-chloro-4-fluorophenyl)-1, 1-dioxido-1,2,6-thiadiazinan-2-yl)acetamido)adamantan-1-carboxamide;
(105) 4-(2-(6-(4-chloro-2-fluorophenyl)-1, 1-dioxido-1,2,6-thiadiazinan-2-yl)acetamido)adamantan-1-carboxamide;
(106) 4-(2-(6-(4-chloro-2-fluorophenyl)-1, 1-dioxido-1,2,6-thiadiazinan-2-yl)acetamido)adamantan-1-carboxamide;
(107) 4-(2-(6-(2,5-dichlorophenyl)-1, 1-dioxido-1,2,6-thiadiazinan-2-yl)acetamido)adamantan-1-carboxamide;
(108) 4-(2-(6-(2-bromophenyl)-1,1-dioxido-1,2,6-thiadiazinan-2-yl)acetamido)adamantan-1-carboxamide;
(109) 4-(2-(1,1-dioxido-6-(2,4,5-trifluorophenyl)-1,2,6-thiadiazinan-2-yl)acetamido)adamantan-1-carboxamide;
(110) 4-(2-(6-(2-chloro-4-fluorophenyl)-1, 1-dioxido-1,2,6-thiadiazinan-2-yl)acetamido)adamantan-1-carboxamide;
(111) 4-(2-(1,1-dioxido-6-(2,3,5,6-tetrafluorophenyl)-1,2,6-thiadiazinan-2-yl)acetamido)adamantan-1-carboxamide;
(112) 4-(2-(1,1-dioxido-6-(2,3,5,6-tetrafluorophenyl)-1,2,6-thiadiazinan-2-yl)acetamido)adamantan-1-carboxamide;
(113) 4-(2-(6-(2,4-dichlorophenyl)-1, 1-dioxido-1,2,6-thiadiazinan-2-yl)acetamido)adamantan-1-carboxamide;
(114) 4-(2-(6-(2,4-dichlorophenyl)-1, 1-dioxido-1,2,6-thiadiazinan-2-yl)acetamido)adamantan-1-carboxamide;
(115) 4-(2-(6-(2-chloro-5-(trifluoromethyl)phenyl)-1, 1-dioxido-1,2,6-thiadiazinan-2-yl)acetamido)adamantan-1-carboxamide;
(116) 4-(2-(6-(2-chloro-5-(trifluoromethyl)phenyl)-1, 1-dioxido-1,2,6-thiadiazinan-2-yl)acetamido)adamantan-1-carboxamide;
(117) 4-(2-(6-(4-chloro-2-(trifluoromethyl)phenyl)-1, 1-dioxido-1,2,6-thiadiazinan-2-yl)acetamido)adamantan-1-carboxamide;
(118) 4-(2-(6-(4-chloro-2-(trifluoromethyl)phenyl)-1, 1-dioxido-1,2,6-thiadiazinan-2-yl)acetamido)adamantan-1-carboxamide;
(119) 4-(2-(6-(2,3-dichlorophenyl)-1, 1-dioxido-1,2,6-thiadiazinan-2-yl)acetamido)adamantan-1-carboxamide;
(120) 4-(2-(6-(2,3-dichlorophenyl)-1, 1-dioxido-1,2,6-thiadiazinan-2-yl)acetamido)adamantan-1-carboxamide;
(121) 4-(2-(6-(2,6-dichlorophenyl)-1, 1-dioxido-1,2,6-thiadiazinan-2-yl)acetamido)adamantan-1-carboxamide;
(122) 4-(2-(1,1-dioxido-7-(2,4,6-trichlorophenyl)-1,2,7-thiadiazepane-2-yl)acetamido)adamantan-1-carboxamide;
(123) 4-(2-(methyl(N-methyl-N-(2,4,6-trichlorophenyl)sulfamoyl)amino)acetamido)adamantan-1-carboxamide;
(124) 4-(2-(1,1-dioxido-5-(2,4,6-trichlorophenyl)-1,2,5-thiadiazolidin-2-yl)acetamido)adamantan-1-carboxamide;
(125) 4-(2-(1,1-dioxido-5-(2,4,6-trichlorophenyl)-1,2,5-thiadiazolidin-2-yl)acetamido)adamantan-1-carboxamide;
(126) 4-(2-(ethyl(N-ethyl-N-(2,4,6-trichlorophenyl)sulfamoyl)amino)acetamido) adamantan-1-carboxamide;
(127) 4-(2-(methyl(N-methyl-N-(2,4,5-trichlorophenyl)sulfamoyl)amino)acetamido)adamantan-1-carboxamide;
(128) 4-(2-(3-methyl-1,1-dioxido-6-(2,4,6-trichlorophenyl)-1,2,6-thiadiazinan-2-yl)acetamido)adamantan-1-carboxamide;
(129) 4-(2-((N-(2-fluorophenyl)-N-methylsulfamoyl)(methyl)amino)acetamido) adamantan-1-carboxamide;
(130) 4-(2-(methyl(N-methyl-N-(2,4,6-trifluorophenyl)sulfamoyl)amino)acetamido)adamantan-1-carboxamide;
(131) 4-(2-(1,1-dioxido-6-(o-tolyl)-1,2,6-thiadiazinan-2-yl)acetamido)adamantan-1-carboxamide;
(132) 4-(2-(6-(2-ethylphenyl)-1, 1-dioxido-1,2,6-thiadiazinan-2-yl)acetamido)adamantan-1-carboxamide;
(133) 4-(2-(6-(3-chloro-2-methylphenyl)-1, 1-dioxido-1,2,6-thiadiazinan-2-yl)acetamido)adamantan-1-carboxamide;
(134) 4-(2-(6-(4-chloro-2-methylphenyl)-1, 1-dioxido-1,2,6-thiadiazinan-2-yl)acetamido)adamantan-1-carboxamide;
(135) 4-(2-(6-(3-fluoro-2-methylphenyl)-1, 1-dioxido-1,2,6-thiadiazinan-2-yl)acetamido)adamantan-1-carboxamide;
(136) 4-(2-(6-(4-fluoro-2-methylphenyl)-1, 1-dioxido-1,2,6-thiadiazinan-2-yl)acetamido)adamantan-1-carboxamide;
(137) 4-(2-(6-(2-fluoro-6-methylphenyl)-1, 1-dioxido-1,2,6-thiadiazinan-2-yl)acetamido)adamantan-1-carboxamide;
(138) 4-(2-(6-(2,6-dichloro-3-methylphenyl)-1, 1-dioxido-1,2,6-thiadiazinan-2-yl) acetamido)adamantan-1-carboxamide;
(139) 4-(2-(1,1-dioxido-6-(3,4,5-trichlorophenyl)-1,2,6-thiadiazinan-2-yl)acetamido)adamantan-1-carboxamide;
(140) 4-(2-(6-(2-chloro-4,6-dimethylphenyl)-1, 1-dioxido-1,2,6-thiadiazinan-2-yl) acetamido)adamantan-1-carboxamide;
(141) 4-(2-(6-(2-fluorophenyl)-1, 1-dioxido-4-((tetrahydro-2H-pyran-2-yl)oxy)-1,2,6-thiadiazinan-2-yl)acetamido)adamantan-1-carboxamide;
(142) 4-(2-(6-(2-fluorophenyl)-4-hydroxy-1,1-dioxido-1,2,6-thiadiazinan-2-yl)acetamido)adamantan-1-carboxamide;
(143) 4-(2-(6-mesityl-1,1-dioxido-1,2,6-thiadiazinan-2-yl)acetamido)adamantan-1-carboxamide;
(144) 4-(2-(6-(2,5-dimethylphenyl)-1, 1-dioxido-1,2,6-thiadiazinan-2-yl)acetamido)adamantan-1-carboxamide;
(145) 4-(2-(6-(2,4-dimethylphenyl)-1, 1-dioxido-1,2,6-thiadiazinan-2-yl)acetamido)adamantan-1-carboxamide;
(146) 4-(2-(6-([1,1′-Biphenyl]-2-yl)-1, 1-dioxido-1,2,6-thiadiazinan-2-yl)acetamido)adamantan-1-carboxamide;
(147) 4-(2-(6-(2-methoxy-6-methylphenyl)-1, 1-dioxido-1,2,6-thiadiazinan-2-yl)acetamido)adamantan-1-carboxamide;
(148) 4-(2-(6-(4-methoxy-2,6-dimethylphenyl)-1, 1-dioxido-1,2,6-thiadiazinan-2-yl)acetamido)adamantan-1-carboxamide;
(149) 4-(2-(6-(2-cyanophenyl)-1, 1-dioxido-1,2,6-thiadiazinan-2-yl)acetamido) adamantan-1-carboxamide;
(150) 4-(2-(6-(2,6-dibromo-4-methylphenyl)-1, 1-dioxido-1,2,6-thiadiazinan-2-yl)acetamido)adamantan-1-carboxamide;
(151) 4-(2-(6-(2,4-dichloro-6-methylphenyl)-1, 1-dioxido-1,2,6-thiadiazinan-2-yl)acetamido)adamantan-1-carboxamide;
(152) 4-(2-(6-(4-bromo-2-chloro-6-methylphenyl)-1, 1-dioxido-1,2,6-thiadiazinan- 2-yl)acetamido)adamantan-1-carboxamide;
(153) 4-(2-(6-(2,4-dimethoxyphenyl)-1, 1-dioxido-1,2,6-thiadiazinan-2- yl)acetamido)adamantan-1-carboxamide;
(154) 4-(2-(6-(2-acetamidophenyl)-1, 1-dioxido-1,2,6-thiadiazinan-2- yl)acetamido)adamantan-1-carboxamide;
(155) 4-(2-(6-(2,3-dihydro-1H-inden-4-yl)-1, 1-dioxido-1,2,6-thiadiazinan-2- yl)acetamido)adamantan-1-carboxamide;
(156) 4-(2-(4-methyl-1, 1-dioxido-6-(2,4,6-trichlorophenyl)-1,2,6-thiadiazinan-2- yl)acetamido)adamantan-1-carboxamide;
(157) 4-(2-(6-(4-bromo-2,6-dimethylphenyl)-1, 1-dioxido-1,2,6-thiadiazinan-2-yl)acetamido)adamantan-1-carboxamide;
(158) 4-(2-(6-(2-bromo-4,6-dimethylphenyl)-1, 1-dioxido-1,2,6-thiadiazinan-2-yl)acetamido)adamantan-1-carboxamide;
(159) 4-(2-(6-(2,6-dibromo-4-fluorophenyl)-1, 1-dioxido-1,2,6-thiadiazinan-2-yl)acetamido)adamantan-1-carboxamide;
(160) 4-(2-(6-(2-bromo-6-chloro-4-fluorophenyl)-1, 1-dioxido-1,2,6-thiadiazinan-2-yl)acetamido)adamantan-1-carboxamide;
(161) 4-(2-(6-(2-bromo-6-chloro-4-fluorophenyl)-1, 1-dioxido-1,2,6-thiadiazinan-2-yl)acetamido)adamantan-1-carboxamide;
(162) 4-(2-(1,1-dioxido-6-(2,4,6-trichlorophenyl)-1,4,2,6-dithiadiazinan-2-yl)acetamido)adamantan-1-carboxamide;
(163) 4-(2-(1,1,4,4-tetraoxido-6-(2,4,6-trichlorophenyl)-1,4,2,6-dithiadiazinan-2-yl)acetamido)adamantan-1-carboxamide;
(164) 4-(2-(4-chloro-1, 1-dioxido-6-(2,4,6-trichlorophenyl)-1,2,6-thiadiazinan-2-yl)acetamido)adamantan-1-carboxamide;
(165) 4-(2-(6-(2-bromo-4-chloro-6-fluorophenyl)-1, 1-dioxido-1,2,6-thiadiazinan-2-yl)acetamido)adamantan-1-carboxamide;
(166) 4-(2-(6-(2-bromo-4,6-dichlorophenyl)-1, 1-dioxido-1,2,6-thiadiazinan-2-yl)acetamido)adamantan-1-carboxamide;
(167) 4-(2-(4-hydroxy-1,1-dioxido-6-(2,4,6-trichlorophenyl)-1,2,6-thiadiazinan-2-yl)acetamido)adamantan-1-carboxamide;
(168) 4-(2-(1,1-dioxido-4-oxo-6-(2,4,6-trichlorophenyl)-1,2,6-thiadiazinan-2-yl)acetamido)adamantan-1-carboxamide;
(169) 4-(2-(4-methoxy-1,1-dioxido-6-(2,4,6-trichlorophenyl)-1,2,6-thiadiazinan-2-yl)acetamido)adamantan-1-carboxamide;
(170) 4-(2-(6-(2,6-dichloro-4-(trifluoromethoxy)phenyl)-1, 1-dioxido-1,2,6-thiadiazinan-2-yl)acetamido)adamantan-1-carboxamide;
(171) 4-(2-(6-(2,6-dichloro-4-(trifluoromethyl)phenyl)-1, 1-dioxido-1,2,6-thiadiazinan-2-yl)acetamido)adamantan-1-carboxamide;
(172) 4-(2-(4,4-difluoro-1,1-dioxido-6-(2,4,6-trichlorophenyl)-1,2,6-thiadiazinan-2-yl)acetamido)adamantan-1-carboxamide;
(173) 4-(2-(4-hydroxy-4-methyl-1,1-dioxido-6-(2,4,6-trichlorophenyl)-1,2,6-thiadiazinan-2-yl)acetamido)adamantan-1-carboxamide;
(174) 4-(2-(4-hydroxy-1,1-dioxido-6-(2,4,6-trichlorophenyl)-4-(trifluoromethyl)-1,2,6-thiadiazinan-2-yl)acetamido)adamantan-1-carboxamide;
(175) 4-(2-(4-methylene-1,1-dioxido-6-(2,4,6-trichlorophenyl)-1,2,6-thiadiazinan-2-yl)acetamido)adamantan-1-carboxamide;
(176) 4-(2-(6,6-dioxido-7-(2,4,6-trichlorophenyl)-6-thia-5,7-diazaspiro[2,5]octane-5-yl)acetamido)adamantan-1-carboxamide;
(177) 4-(2-(4,4-dimethyl-1,1-dioxido-6-(2,4,6-trichlorophenyl)-1,2,6-thiadiazinan-2-yl) acetamido)adamantan-1-carboxamide;
(178) 4-(2-(6-(2-chloro-4-(trifluoromethyl)phenyl)-1, 1-dioxido-1,2,6-thiadiazinan-2-yl)acetamido)adamantan-1-carboxamide;
(179) 4-(2-(6-(4-bromo-2-(chlorophenyl)-1, 1-dioxido-1,2,6-thiadiazinan-2-yl)acetamido)adamantan-1-carboxamide;
(180) 4-(2-(6-(2-bromo-4-(chlorophenyl)-1, 1-dioxido-1,2,6-thiadiazinan-2-yl)acetamido)adamantan-1-carboxamide;
(181) 4-(2-(4-methyl-1,1-dioxido-6-phenyl-1,2,6-thiadiazinan-2-yl)acetamido)adamantan-1-carboxamide;
(182) 4-(2-(6-(2-bromo-4-chloro-6-fluorophenyl)-4-methyl-1, 1-dioxido-1,2,6-thiadiazinan-2-yl)acetamido)adamantan-1-carboxamide;
(183) 4-(2-(6-(2,6-dichloro-4-(trifluoromethyl)phenyl)-4-methyl-1, 1-dioxido-1,2,6-thiadiazinan-2-yl)acetamido)adamantan-1-carboxamide;
(184) 4-(2-(6-(2,6-dichloro-4-(trifluoromethyl)phenyl)-4-methylene-1,1-dioxido-1,2,6-thiadiazinan-2-yl)acetamido)adamantan-1-carboxamide;
(185) 4-(2-(4-methylene-1,1-dioxido-6-phenyl-1,2,6-thiadiazinan-2-yl)acetamido)adamantan-1-carboxamide;
(186) 4-(2-(6-(2-bromo-4-chloro-6-fluorophenyl)-4-methylene-1,1-dioxido-1,2,6-thiadiazinan-2-yl)acetamido)adamantan-1-carboxamide;
(187) 4-(2-(6-(4-chloro-2-iodophenyl)-4-methyl-1,1-dioxido-1,2,6-thiadiazinan-2-yl)acetamido)adamantan-1-carboxamide;
(188) 4-(2-(6-(2-iodophenyl)-4-methyl-1, 1-dioxido-1,2,6-thiadiazinan-2-yl)acetamido)adamantan-1-carboxamide;
(189) 4-(2-(1,1-dioxido-4-oxo-5-(2,4,6-trichlorophenyl)-1,2,5-thiadiazolidin-2-yl) acetamido)adamantan-1-carboxamide;
(190) 4-(3-(4-methyl-1,1-dioxido-6-(2,4,6-trichlorophenyl)-1,2,6-thiadiazinan-2-yl)propaneamido)adamantan-1-carboxamide;
(191) 4-(2-(1,1-dioxido-5-oxo-6-(2,4,6-trichlorophenyl)-1,2,6-thiadiazinan-2-yl)acetamido)adamantan-1-carboxamide;
(192) 4-(2-(6-(2-chloro-4-nitrophenyl)-4-methyl-1,1-dioxido-1,2,6-thiadiazinan-2-yl)acetamido)adamantan-1-carboxamide;
(193) 4-(2-(6-(4-chloro-2-nitrophenyl)-4-methyl-1,1-dioxido-1,2,6-thiadiazinan-2-yl)acetamido)adamantan-1-carboxamide;
(194) 4-(2-(2,2-dioxidobenzo[c][1,2,5]thiadiazol-1(3H)-yl)acetamido)adamantan-1-carboxamide;
(195) 4-(2-((S)-6-(2,6-dichloro-4-(trifluoromethyl)phenyl)-4-methyl-1, 1-dioxido-1,2,6-thiadiazinan-2-yl)acetamido)adamantan-1-carboxamide;
(196) 4-(2-((R)-6-(2,6-dichloro-4-(trifluoromethyl)phenyl)-4-methyl-1,1-dioxido-1,2,6-thiadiazinan-2-yl)acetamido)adamantan-1-carboxamide;
(197) 4-(2-(6-(2-chloro-4-(methylsulfonamido)phenyl)-4-methyl-1,1-dioxido-1,2,6-thiadiazinan-2-yl)acetamido)adamantan-1-carboxamide;
(198) 4-(2-(6-(4-acetamido-2-chlorophenyl)-4-methyl-1, 1-dioxido-1,2,6-thiadiazinan-2-yl)acetamido)adamantan-1-carboxamide;
(199) 4-(2-(6-(2-chloro-4-(3-ethylureido)phenyl)-4-methyl-1, 1-dioxido-1,2,6-thiadiazinan-2-yl)acetamido)adamantan-1-carboxamide;
(200) 4-(2-(1,1-dioxido-7-(2,4,6-trichlorophenyl)-6,7-dihydro-1,2,7-thiadiazepine-2(3H)-yl)acetamido)adamantan-1-carboxamide;
(201) 4-(2-(allyl(N-allyl-N-(2,6-dichloro-4-(trifluoromethyl)phenyl)sulfamoyl)amino)acetamido)adamantan-1-carboxamide;
(202) 4-(2-(6-(2,6-dichloro-4-(trifluoromethyl)phenyl)-4-isopropyl-1,1-dioxido-1,2,6-thiadiazinan-2-yl)acetamido)adamantan-1-carboxamide;
(203) 4-(2-((S)-4-methyl-1,1-dioxido-6-(2,4,6-trichlorophenyl)-1,2,6-thiadiazinan-2-yl)acetamido)adamantan-1-carboxamide;
(204) 4-(2-((R)-4-methyl-1,1-dioxido-6-(2,4,6-trichlorophenyl)-1,2,6-thiadiazinan-2-yl)acetamido)adamantan-1-carboxamide;
(205) 4-(2-(6-(2,6-dichloro-4-(trifluoromethyl)phenyl)-4-ethyl-1, 1-dioxido-1,2,6-thiadiazinan-2-yl)acetamido)adamantan-1-carboxamide;
(206) 4-(2-(4-methyl-1, 1-dioxido-6-(pyridine-2-yl)-1,2,6-thiadiazinan-2-yl)acetamido)adamantan-1-carboxamide;
(207) 4-(2-(4-methyl-6-(5-nitropyridine-2-yl)-1, 1-dioxido-1,2,6-thiadiazinan-2-yl)acetamido)adamantan-1-carboxamide;
(208) 4-(2-(4-methyl-1, 1-dioxido-6-(5-(trifluoromethyl)pyridine-2-yl)-1,2,6-thiadiazinan-2-yl)acetamido)adamantan-1-carboxamide;
(209) 4-(2-(6-(4-bromo-2,6-dichlorophenyl)-4-methyl-1, 1-dioxido-1,2,6-thiadiazinan-2-yl)acetamido)adamantan-1-carboxamide;
(210) 4-(2-(6-(3,5-dichloro-[1, 1′-biphenyl]-4-yl)-4-methyl-1, 1-dioxido-1,2,6-thiadiazinan-2-yl)acetamido)adamantan-1-carboxamide;
(211) 4-(2-(6-(3,5-dichloro-2′,4′-difluoro-[1, 1′-biphenyl]-4-yl)-4-methyl-1, 1-dioxido-1,2,6-thiadiazinan-2-yl)acetamido)adamantan-1-carboxamide;
(212) 4-(2-(6-(2,6-dichloro-4-(puran-2-yl)phenyl)-4-methyl-1, 1-dioxido-1,2,6-thiadiazinan-2-yl)acetamido)adamantan-1-carboxamide;
(213) 4-(2-(6-(2,6-dichloro-4-cyanophenyl)-4-methyl-1, 1-dioxido-1,2,6-thiadiazinan-2-yl)acetamido)adamantan-1-carboxamide;
(214) 4-(2-(6-(2,6-dichloro-4-methylphenyl)-4-methyl-1, 1-dioxido-1,2,6-thiadiazinan-2-yl)acetamido)adamantan-1-carboxamide;
(215) 4-(2-(6-(2,6-dichloro-4-propylphenyl)-4-methyl-1, 1-dioxido-1,2,6-thiadiazinan-2-yl)acetamido)adamantan-1-carboxamide; and
(216) 4-(2-(6-(2,6-dichloro-4-cyclopropylphenyl)-4-methyl-1, 1-dioxido-1,2,6-thiadiazinan-2-yl)acetamido)adamantan-1-carboxamide.

According to another aspect of the present invention, Formula 1 can be (1s, 3R,4s,5S,7s)-4-(2-(6-(2,6-dichloro-4-(trifluoromethyl)phenyl)-4-methyl-1,1-dioxido-1,2,6-thiadiazinan-2-yl)acetamido)adamantane-1-carboxamide (hereinafter KR-67607). In the present invention, 4-(2-(6-(2,6-dichloro-4-(trifluoromethyl)phenyl)-4-methyl-1,1-dioxido-1,2,6-thiadiazinan-2-yl)acetamido)adamantane-1-carboxamide may be in E-form or Z-form, for example, E-form.

KR-67607 is known to have good inhibitory activity against 11β-HSD1. However, while several attempts have been made to develop it into an ophthalmic formulation for therapeutic effectiveness and patient compliance, its very poor solubility in water (<0.1 ug/mL) has limited the development of ophthalmic formulations for the treatment of glaucoma, particularly in solution.

In the present invention, pharmaceutically acceptable salts of Formula 1 include acid addition salts formed with pharmaceutically acceptable free acids.

The acid addition salts may be formed using inorganic acids such as hydrochloride, nitric acid, phosphoric acid, sulfuric acid, hydrobromic acid, hydroiodic acid, nitrous acid and phosphorous acid; or organic acids aliphatic mono- and dicarboxylate, phenyl-substituted alkanoate, hydroxy alkanoate and alkandioate, aromatic acids, nontoxic organic acids such as aliphatic and aromatic sulfonic acids, acetic acid, benzoic acid, citric acid, lactic acid, maleic acid, gluconic acid, methanesulfonic acid, 4-toluenesulfonic acid, tartaric acid and fumaric acid. Such pharmaceutically nontoxic salts include sulfate, pyrosulfate, bisulfate, sulfite, bisulfate, nitrate, phosphate, monohydrogen phosphate, dihydrogen phosphate, metaphosphate, pyrophosphate chloride, bromide, iodide, fluoride, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexane-1,6-dioate, benzoate, chlorobenzoate, methylbenzoate, dinitro benzoate, hydroxybenzoate, methoxybenzoate, phthalate, terephthalate, benzene sulfonate, toluene sulfonate, chlorobenzene sulfonate, xylenesulfonate, phenyl acetate, phenylpropionate, phenylbutyrate, citrate, lactate, hydroxybutyrate, glycolate, malate, tartrate, methane sulfonate, propane sulfonate, naphthalene-1-sulfonate, naphthalene-2-sulfonate or mandelate. For example, it may be, but is not limited to, hydrochloride, sulfate, acetate, trifluoroacetate, phosphate, fumarate, maleate, citrate, methanesulfonate, or lactate.

In the present invention, the acid addition salts may be prepared according to conventional methods, for example, by dissolving a compound of Formula 1 in an organic solvent and adding an organic or inorganic acid and filtering and drying the resulting precipitate, or by distilling the solvent and excess acid under reduced pressure and then drying or crystallizing in an organic solvent.

In the present invention, the organic solvent may be, but is not limited to, methanol, ethanol, acetone, methylene chloride, acetonitrile, and the like.

In the present invention, the salt may be a pharmaceutically acceptable metal salt prepared using a base, for example, but not limited to, an alkali metal or alkaline earth metal salt.

In the present invention, an alkali metal or alkaline earth metal salt may be obtained by dissolving the compound in an excess of an alkali metal hydroxide or alkaline earth metal hydroxide solution, filtering the non-dissolved compound salt, evaporating the filtrate, and drying, but is not limited thereto.

In the present invention, it is pharmaceutically suitable to prepare sodium, potassium, or calcium salts, but is not limited thereto. Further, the corresponding silver salt may be obtained by reacting an alkali metal or alkaline earth metal salt with a suitable silver salt, for example, silver nitrate.

### [Cyclodextrin or Cyclodextrin Derivatives]

Molecular Inclusion Encapsulation is a technique for water-solubilizing poorly soluble drugs by enclosing them in carbohydrates such as cyclodextrin (CD).

The present invention uses cyclodextrin or a derivative thereof as a solubilizer for molecular-scale nano-inclusion to solubilize a poorly water-soluble drug of Formula 1 for delivery through the comeal epithelium into the ocular tissue. In this case, the cyclodextrin or derivative thereof can also serve as a carrier in body fluids for the poorly soluble drug of Formula 1.

In the pharmaceutical field, CDs can be used as carriers, solubilizers, and adjuvants such as derivatives. Usually, drugs complexed with CDs are transported to the stomach in an aqueous phase much faster than normal drugs and are dissociated and absorbed in the stomach.

When orally administering poorly soluble drugs, complexation with CDs can make them more soluble in the blood, and CD complexation can reduce the hydrophobicity of poorly soluble drugs.

Cyclodextrin (CD) is a cyclic, non-reducing maltooligosaccharide with glucose molecules linked by α-1,4-glucosidic bonds, produced by cyclodextrin glucanotransferase (CGTase) from starch, amylose, amylopectin, dextrin, glycogen, and long-chain maltooligosaccharides as substrates. CD is a crystalline, non-hygroscopic substance that is shaped like a doughnut with six, seven, and eight glucose molecules connected in a ring, respectively, and is referred to as α-CD, β-CD, and γ-CD depending on their number. β-CD is thermodynamically stable compared to other types, and many strains secrete enzymes that produce it, making it the most economical CD for industrial use, but it has the disadvantage of low solubility in water.

CDs have an inner cavity due to their cyclic ring structure, and the hydroxyl group at the C6 position, which is exposed to the outside of each glucose, is hydrophilic, while the inside is hydrophobic due to hydrogen bonds and ether bonds. Therefore, because the inside of the CD has a certain size of empty space and the outside is hydrophilic, various hydrophobic substances can be enclosed in the ring space, and the solubility can be increased due to the hydrophilic outside.

When a hydrophobic substance is added from the outside, the CD acts as a host, enclosing the foreign substance in the cavity to form an inclusion complex, which can play a role in protecting and stabilizing the enclosed guest substance according to these properties.

The most important factor affecting CDs' ability to form complexes is the stereostructural characteristics of the guest molecule. In other words, the inner diameters of the cavities of α-CD, β-CD, and γ-CD are different, so compounds with molecular structures suitable for each cavity are specifically entrapped. In addition, external environmental conditions such as polarity, charge, temperature, and ionic strength of the guest molecule are also important factors. The binding forces for complex formation include hydrophobic effects, van der Waals bonds, hydrogen bonding, energy reduction by the release of high-energy molecules in the CD cavity, and the release of strain energy in the cyclic structure of the CD by the binding of the ligand.

In addition, CDs do not cause any toxicity such as carcinogenicity or mutagenicity, and are easily absorbed and metabolized in mammals and humans. Unlike starch, which is degraded in the small intestine, CD is absorbed in small amounts in the stomach and small intestine, and is mostly degraded by the microbiota in the colon and released as carbon dioxide and water.

α-, β-, and γ-CD s are the first generation of CDs (or parent CDs), and as the range of applications of parent CDs expanded, second generation CDs or CD derivatives with more specialized forms and functions were developed. CD derivatives are forms in which various types of substituents are attached to the parent CD through enzymatic or chemical methods. CD derivatives include branched CDs, chemically modified CD derivatives, and CD polymers. Most of these derivatives have improved properties, such as entrapment capacity and solubility, over the natural state of CD.

Methylation of CD increases the solubility and stability of complexes with hydrophobic materials.

Among the cyclodextrin derivatives with better physical properties and entrapment capacity than natural cyclodextrin, 2-hydroxypropyl-β-cyclodextrin (HP-β-CD) has a higher solubility in water and lower toxicity, i.e., β-cyclodextrin has a solubility of 18.5 mg/ml in water, but HP-β-CD has a solubility of more than 1 g/ml, and unlike natural cyclodextrin, it is not hemolytic, so it is often used in injectable formulations.

### [Ophthalmic Formulations Containing Inclusion Complexes of Poorly Soluble Drugs]

Ophthalmic formulations, or eye-drops, are medicinal solutions that are instilled into the eye and applied to the conjunctival sac. Since they contact the sensitive mucous membrane of the eye, their osmolarity and pH should be similar to tears. The change in pH is not only related to irritation, but also affects the degree of ionization, for which buffers are added to increase the non-ionized part and make it easier to pass through the corneal epithelium.

In clinical practice, the goal of ocular applications is to deliver a targeted dose of the drug to the desired ocular tissue without damaging normal tissue. However, the first factor that affects drug absorption is tears, and when eye drops are dropped into the eye, they first mix with the tears in front of the cornea, and only a very small portion of the drops are absorbed into the eye. Typical eye drops come in liquid form with a low viscosity.

The normal amount of tears present is approximately 7 to 10 μl, with 1 μl covering each membrane and 3 to 4 μl in the upper and lower conjunctival sacs. A drop of commercialized ophthalmic formulations averages about 40 μl (25 to 70 μl), but because the eye can only hold about 25 to 30 μl of liquid at a time, much of it is lost through the nasolacrimal duct in 15 to 30 seconds. In addition, turnover from normal tear production also contributes to the elimination of ophthalmic formulations, with turnover rates in the unstimulated eye measured to be on the order of 1 μl/min. Based on this, it can be assumed that instilled ophthalmic formulations take approximately 10 minutes to completely drain through the nasolacrimal duct in front of the cornea. Therefore, solubilizers and solubilization methods are urgently needed to ensure that the poorly soluble drug of formula 1 can pass through the corneal epithelium and be delivered into the ocular tissue in sufficient concentration. Furthermore, solubilization techniques can significantly increase the permeation rate and bioavailability of poorly soluble drugs and the residence time of the drug in the cornea.

As will be described later, the prerequisites for the discovery in Examples 31 through 35 that the poorly soluble drug of Formula 1 can inhibit cortisol synthesis by inhibiting the activity of 11β-HSD1 and thereby strongly protect the optic nerve and other intraocular tissues are (1) the development of a solubilization method for the poorly soluble drug of Formula 1 and its preparation into an eye drop formulation, and (2) the ability of the eye drop formulation to be smoothly delivered to the retina and optic nerve tissues of the eye when administered by eye drops.

In view of these considerations, the present invention provides an ophthalmic formulation in the form of an eye drop comprising an inclusion complexes, in which a poorly water-soluble drug of Formula 1 has been entrapped in cyclodextrin or a cyclodextrin derivative in an aqueous solution of pH 10 or higher for delivery through the corneal epithelium and into the ocular tissues.

The ophthalmic formulation according to the present invention can be provided by a solubilization method comprising the steps below:
a step of preparing a solution in which a solubilization promoter is dissolved in water to obtain a solution having a pH of at least 10.0;
a solubilizer dissolution step in which the solution is mixed with cyclodextrin or a cyclodextrin derivative as a solubilizer;
a compound dissolving step comprising mixing in the solution a compound represented by Formula 1 or a pharmaceutically acceptable salt thereof; and
a pH adjustment step comprising mixing a pH adjusting agent into the solution.

In the present invention, the method may further comprise the step of dissolving in the solution at least one selected from the group consisting of buffers, isotonic agents, viscosity modifiers, antioxidants, and chelating agents.

In the present invention, the solubilization promoter may be at least one selected from the group consisting of a basic substance and a buffer, but is not limited thereto, and may be any substance that can be used to obtain a solution of pH 10 or higher.

In the present invention, there is no restriction on the use of water, but it is preferably free of salts or other ions.

In the present invention, cyclodextrins or cyclodextrin derivatives may be at least one selected from the group consisting of α-cyclodextrin, β-cyclodextrin, γ-cyclodextrin, methyl-substituted cyclodextrin, ethyl-substituted cyclodextrin, alkyl ether cyclodextrin, 2-hydroxypropyl-β-cyclodextrin, sulfobutyl ether-β-cyclodextrin, hydroxyethyl-β-cyclodextrin, and 2-hydroxypropyl-γ-cyclodextrin, and may be, for example, 2-hydroxypropyl-β-cyclodextrin.

In the present invention, the weight ratio of the compound represented by Formula 1, or a pharmaceutically acceptable salt thereof, to the solubilizer may be from 1:10 to 40, for example, from 1:15 to 25. At ratios of 1:10 or less, it is difficult to prepare clear solutions to therapeutic concentrations of the compounds represented by Formula 1 due to insufficient solubilization, and at ratios greater than 1:40, it is difficult to prepare clear solutions due to solubility limitations of the solubilizer.

The compounds represented by Formula 1, or pharmaceutically acceptable salts thereof, in the ophthalmic formulation of the present invention, as finally prepared, may be in an amount of 0.01 to 1.0 w/v %, 0.02 to 1.0 w/v %, 0.03 to 1. 0 w/v %, 0.04 to 1.0 w/v %, 0.05 to 1.0 w/v %, 0.01 to 0.9 w/v %, 0.02 to 0.9 w/v %, 0.03 to 0.9 w/v %, 0.04 to 0.9 w/v %, 0.05 to 0.9 w/v %, 0.01 to 0.8 w/v %, 0.02 to 0.8 w/v %, 0.03 to 0. 8 w/v %, 0.04 to 0.8 w/v %, 0.05 to 0.8 w/v %, 0.01 to 0.7 w/v %, 0.02 to 0.7 w/v %, 0.03 to 0.7 w/v %, 0.04 to 0.7 w/v %, 0.05 to 0.7 w/v %, 0.01 to 0.6 w/v %, 0.02 to 0.6 w/v %, 0.03 to 0. 6 w/v %, 0.04 to 0.6 w/v %, 0.05 to 0.6 w/v %, 0.01 to 0.5 w/v %, 0.02 to 0.5 w/v %, 0.03 to 0.5 w/v %, 0.04 to 0.5 w/v %, and may be present in concentrations ranging from 0.05 to 0.5 w/v %, for example.

In the present invention, the buffer may be, but is not limited to, phosphoric acid and salts thereof, boric acid and salts thereof, and citric acid and salts thereof.

In the present invention, the isotonic agent may be, but is not limited to, sodium chloride, mannitol, sorbitol, glycerin, and the like.

In the present invention, the pH adjusting agent may be, but is not limited to, hydrochloric acid, sulfuric acid, sodium hydroxide, potassium hydroxide, and the like.

In the present invention, the viscosity regulator may be at least one selected from the group consisting of polyvinyl alcohol, polyvinyl pyrrolidone, methyl cellulose, hydroxypropyl methyl cellulose, hydroxyethyl cellulose, carboxymethyl cellulose, and hydroxypropyl cellulose, and may be, for example, polyvinyl pyrrolidone. They have good solubility in water, are easy to adjust viscosity depending on the type (K12, K17, K30, K90, etc.), and have a dissolution aid effect for some substances.

In the present invention, antioxidants are sodium sulfite, sodium sulfate, sodium bisulfite, sodium metabisulfite, sodium ascorbate, tocopherol, butylated hydroxy anisole (BHA) and dibutyl hydroxy toluene (BHT), and may be one or more selected from the group consisting of, but not limited to.

In the present invention, the chelating agent may be, but is not limited to, ethylenediaminetetraacetic acid (EDTA).

To reduce ocular irritation, ophthalmic formulations should have an osmolarity similar to that of normal tears of 300 mOsm/kg. The osmolarity that the eye can tolerate ranges from 200 to 600 mOsm/kg or 0.2% to 2.0% NaCl. Another factor that affects this irritation is pH. The pH should be maintained between 4.5 and 9 to reduce the irritation, but if the discomfort increases after the administration of the eye drops, the amount of tears increases after the administration and the eye blinks a lot, so whether the eye is irritated or not will affect the absorption of the medicine.

Therefore, in the present invention, the step of adjusting the pH may be a step of adjusting the pH of the solution to 5 to 9, 6 to 9, 5 to 8, 6 to 8, for example, adjusting it to pH 7. The least irritating to the eyes is within the above range.

Furthermore, the osmolarity of the ophthalmic formulation according to the present invention as finally prepared may be 250 to 340 mosmol, 260 to 340 mosmol, 270 to 340 mosmol, 280 to 340 mosmol, 250 to 330 mosmol, 260 to 330 mosmol, 270 to 330 mosmol, 280 to 330 mosmol, 250 to 320 mosmol, 260 to 320 mosmol, 270 to 320 mosmol, 280 to 320 mosmol, 250 to 310 mosmol, 260 to 310 mosmol, 270 to 310 mosmol, 280 to 310 mosmol, 250 to 300 mosmol, 260 to 300 mosmol, 270 to 300 mosmol, and may be, for example, 280 to 300 mosmol. The above ranges have the advantage of causing less irritation, such as pain, with the isotonic fluid.

Thus, the water insoluble drug(Formula 1) - containing ophthalmic formulation in the form of an eye drop provided in accordance with the present invention can pass through the corneal epithelium and be delivered into the ocular tissue to protect cells from ischemic injury according to the intended mechanism of action (inhibition of cortisol production ->activation of Nrf2/HO-1 signaling pathway ->protection of cells/tissues from ischemic injury).

[Pharmacodynamics of a Poorly Soluble Drug of Formula 1 when Administered as an Eye Drop Formulation].

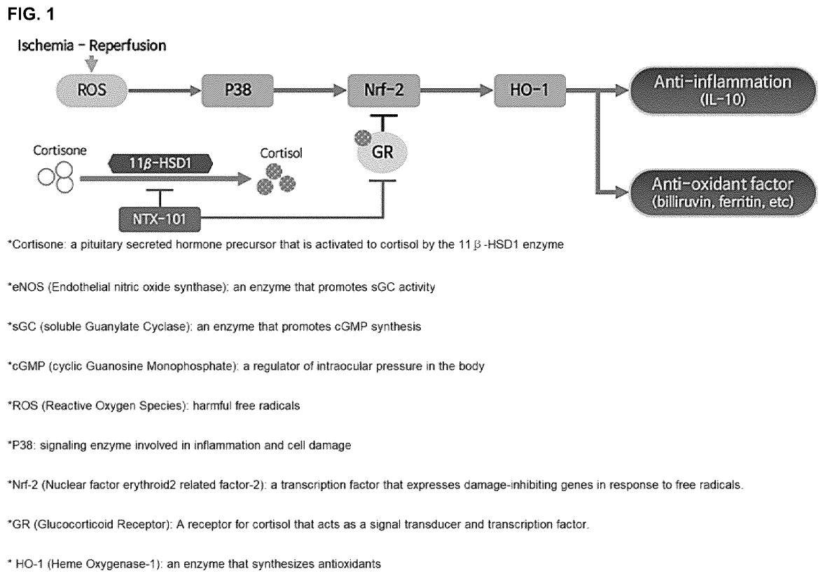

Existing commercialized glaucoma drugs mostly focus on intraocular pressure-lowering effects. In contrast, the ophthalmic formulation in the form of an eye drop containing the poorly soluble drug of Formula 1 according to the present invention passes through the corneal epithelium and is delivered into the ocular tissue, where it inhibits the increase in intraocular pressure by inhibiting an enzyme (11β-HSD1) related to cortisol, a hormone that increases intracellular intraocular pressure, while protecting the optic nerve by activating the antioxidant factor Nrf2/HO-1 (FIG. 1). Thus, the target of action of the poorly soluble drug of Formula 1, an inhibitor of the 11β-HSD1 enzyme, is intracellular 11β-HSD1 distributed in the aqueous humor-producing tissues of the eye (specifically, the retina) and optic nerve. In other words, through an eye drop formulation designed to pass through the corneal epithelium and be delivered into the ocular tissues according to the present invention, the poorly soluble drug of Formula 1 can protect the ocular tissues and/or optic nerve by a mechanism of inhibition of intracellular cortisol production and/or activation of the antioxidant factor Nrf2/HO-1.

The present invention confirms that the poorly soluble drug of Formula 1 is a candidate for ophthalmic treatment with optic nerve protection against ischemic optic nerve diseases such as glaucoma, which can protect the optic nerve by inhibiting ischemic damage to ocular tissues by inhibiting the well-established target 11β-HSD1. Further, various in vivo animal experiments have confirmed its pharmacokinetic properties in terms of passing through the corneal epithelium into ocular tissues (Example 31) and its pharmacological efficacy in terms of lowering intraocular pressure and protecting the optic nerve (Example 32 to Example 34).

Intraocular pressure is the pressure in the eye that maintains the shape of the eyeball, and elevated intraocular pressure is caused by an imbalance in the production and outflow of aqueous humor. Aqueous humor is generated from the ciliary body, fills the posterior chamber, passes through the pupil of the iris, and fills the anterior chamber, which is bordered by the iris, and the posterior chamber is bordered by the cornea and lens. The aqueous humor then exits through the trabecular meshwork and the uveoscleral pathway.

Ischemic optic neuropathy, such as glaucoma (including normal-tension glaucoma), is a disease in which ischemic damage occurs to the retina and optic nerve cells, affecting vision and visual acuity.

According to the pathophysiology of ischemic optic neuropathy, (1) ischemia-reperfusion damage occurs to optic nerve/retinal cells due to various causes, (2) various inflammatory responses, autoimmune responses, etc. appear as a result, and (3) further optic nerve damage progresses more rapidly. Therefore, in order to prevent optic nerve damage, it is very important to block the first stage of ischemia-reperfusion injury.

When ischemic optic nerve damage occurs, tissue death centered on the offending cells occurs, and this abnormal tissue death induces a variety of inflammatory responses.

The inflammatory response spreads to surrounding microglia, astrocytes, and other tissues, which release pro-inflammatory cytokines, leading to further inflammatory responses and, in severe cases, the induction of autoimmune responses that attack autologous nerve cells.

In addition, in response to peripheral cell death, astrocytes and other surrounding tissues release various stimuli that can induce neuronal death, and changes occur that inhibit the release of neurotrophins that repair damage and prevent neuronal death, leading to a state in which optic nerve cells die more rapidly in response to small injuries.

Therefore, in order to inhibit this chain reaction of optic nerve damage, it is very important to protect the tissue from ischemia-reperfusion injury caused by various causes.

The body already has a natural defense mechanism against ischemia-reperfusion injury. The Nrf2/HO-1 pathway, which operates in all tissues of the body, is a powerful defense mechanism against ischemic optic nerve injury and acts simultaneously as an antioxidant/anti-inflammatory, allowing the body's normal tissues to maintain their normal state despite small ischemia-reperfusion injuries.

In other words, when ischemia-reperfusion damage occurs, the transcription factor Nrf2 is activated and translocated to the nucleus, where it induces the expression of various antioxidants and anti-inflammatory substances to overcome the damage.

It has already been established that Nrf2 can be activated in the eye to overcome ischemic optic nerve damage. Many preclinical studies have demonstrated that ischemic optic nerve injury can be overcome by intraocular injection of substances that directly activate Nrf2.

However, because over-activation of Nrf2 is an established risk factor for cancer and other immune diseases, there are limitations to developing therapeutic agents that induce artificial over-activation of Nrf2 in ischemic optic neuropathy, which requires long-term treatment. Therefore, there is a need to develop a method to restore the activity of Nrf2, which is significantly reduced in the optic nerve tissue of patients with ischemic optic neuropathy, to normal levels to secure neuroprotective effects.

In the human body, cortisol exists as a natural stress response hormone, and glucocorticoid steroid substances such as cortisol are potent inhibitors of Nrf2 activation.

Cortisol is secreted from the pituitary gland as the inactive precursor cortisone, which is then sent to each localized tissue in the body. In each tissue, cortisone is then converted to cortisol by the enzyme 11β-HSD1, and the amount of cortisol in each tissue is regulated. Each tissue in the body responds to stress by adjusting the activity of the 11β-HSD1 enzyme to regulate the amount of cortisone converted to cortisol. When tissues in the body are highly stressed, the activity of 11β-HSD1 increases, leading to higher concentrations of cortisol, which in turn inhibits Nrf2 activation. Tissues with reduced Nrf2 activation, unlike normal tissues, are unable to respond to ischemia-reperfusion injury after which Nrf2 is unable to counteract the damage.

In tissues with ischemic optic nerve injury, there is an excess of the stress response hormone cortisol due to the intense stress. In the presence of excess cortisol, the activity of Nrf2 is reduced, and the Nrf2/HO-1 pathway cannot be activated normally, making it highly vulnerable to ischemia-reperfusion injury.

Experiments have shown that in eyes subjected to various ischemic optic nerve injuries, the activity of the 11β-HSD1 enzyme is elevated and cortisol is present at significantly higher concentrations than in normal tissues, resulting in a state of impaired activation of Nrf2. Furthermore, in an animal model in which sustained ischemic damage to the eye is induced, we demonstrated that inhibiting the activity of the 11β-HSD1 enzyme reduces the concentration of cortisol and restores the activity of the Nrf2/HO-1 pathway to normal levels, thereby inhibiting the development of ischemic damage and protecting eye tissue and function. This confirms that the mechanism of action of restoring Nrf2 to its normal functional level, rather than abnormally activating Nrf2, to induce tissue protection, can maintain optic nerve protection efficacy without severe side effects even in long-term use.

In conclusion, the present invention establishes a mechanism of action to protect ocular tissues from ischemia-reperfusion injury by regulating the activity of 11β-HSD1 to normalize cortisol concentrations in the retina and optic nerve tissues in the eye and reactivate the Nrf2/HO-1 pathway, and is expected to exert a strong optic neuroprotective effect without side effects.

In short, patients with ischemic optic neuropathy are characterized by increased activity of 11β-HSD1 in the eye, resulting in elevated cortisol concentrations, and when ischemic damage occurs to the optic nerve, the body's ability to protect optic nerve tissue from damage is inhibited, resulting in worse optic nerve damage than normal people and ultimately leading to blindness. Therefore, in the present invention, the target of action of the poorly soluble drug of Formula 1 is 11β-HSD1, which is distributed in the intraocular water-producing tissue and optic nerve, so that the poorly soluble drug of Formula 1 can protect the optic nerve and other intraocular tissue structures by inhibiting the synthesis of cortisol, which aggravates tissue damage in the situation of ischemic injury, through inhibition of the activity of 11β-HSD1. Concomitantly, active inhibition of 11β-HSD1 inhibits aqueous humor production and inhibits fibrosis of aqueous humor draining tissues, thereby lowering intraocular pressure (FIG. 1).

### [Pharmaceutical Compositions]

The pharmaceutical compositions of the present invention can be administered orally or parenterally, e.g., intraocularly.

The formulation of the pharmaceutical composition of the present invention may be an ophthalmic formulation in the form of an eye drop.

The pharmaceutical compositions of the present invention may be, but are not limited to, in the form of an aqueous, clear solution.

In the treatment of ischemic optic neuropathy (e.g., glaucoma), two or three drugs of different classes may be used in combination if a single susceptibility inclusion complex comprising a cyclodextrin or a cyclodextrin derivative entrapping a poorly soluble drug of Formula 1 is insufficient. In this case, two or more single-agent drugs can be administered separately, or a combination of the two can be provided. The combination may eliminate the need to wait to add a second medication, and may prevent overflow and loss of some medication from the conjunctival sac due to adding two medications at once.

The pharmaceutical compositions of the present invention may comprise any pharmaceutically acceptable carrier.

Pharmaceutically acceptable carriers for inclusion in the pharmaceutical compositions of the present invention are those commonly utilized in pharmaceutical formulations, such as lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methylhydroxybenzoate, propylhydroxybenzoate, talc, magnesium stearate, and mineral oil.

In addition to the above ingredients, the pharmaceutical compositions of the present invention may further comprise lubricants, wetting agents, sweeteners, flavoring agents, emulsifiers, suspending agents, preservatives, and the like. Suitable pharmaceutically acceptable carriers and formulations are described in detail in Remington's Pharmaceutical Sciences (19th ed., 1995).

Suitable dosages of the pharmaceutical compositions of the present invention may be prescribed in a variety of ways depending on factors such as the method of formulation, mode of administration, patient age, weight, sex, medical condition, food, time of administration, route of administration, rate of excretion, and response sensitivity. The daily dose of a pharmaceutical composition of the present invention may be, for example, but not limited to, 0.001 to 100 mg/kg. For eye drop formulations, the dosage may be 5 to 15 μl to minimize loss of ophthalmic solution due to tear drainage.

The present invention has developed a method of solubilizing a poorly water-soluble drug of Formula 1 for delivery through the corneal epithelium and into the ocular tissues to provide a clear, transparent, solution-like pharmaceutical composition for administration in an eye drop formulation to a patient in need of optic nerve protection.

The ophthalmic formulation in the form of an eye drop containing the water-insoluble drug of Formula 1 of the present invention is expected to have a differentiated efficacy compared to conventional drugs because it is able to pass through the corneal epithelium and deliver into the ocular tissues to normalize the Nrf2/HO-1 pathway activity through inhibition of the activity of intracellular 11β-HSD1, which is a mechanism of action that exhibits optic nerve protection, thereby blocking the development of ischemia-reperfusion injury in the pathophysiology of ischemic optic neuropathy. In addition, it has a differentiated performance compared to existing therapies through its direct optic nerve protection effect when administered by eye drops. It has a relatively safe mechanism of action in terms of systemic side effects, making it a possible treatment for patients who are non-compliant with existing treatments.

Hereinafter, the present invention will be described in more detail through examples. These examples are for illustrative purposes only, and it will be apparent to those of ordinary skill in the art that the scope of the present invention is not construed as being limited by these examples.

### Preparation Example 1. Preparation of a Solution with pH 1

After mixing 750 ml of 0.1 M HCl aqueous solution (Solution A) and 250 ml of 0.1 M KCl aqueous solution (Solution B), small amounts of Solutions A and B were added dropwise to adjust the pH to 1.

Mix 750 mL of 0.1 M HCl aqueous solution (solution A) and 250 mL of 0.1 M KCl aqueous solution (solution B), then add small amounts of solution A and B to adjust the pH to 1.

### Preparation Example 2. pH 3 Solution Preparation

After mixing 790 mL of 0.1 M citric acid aqueous solution (Solution A) and 900 mL of 0.2 M Na₂HPO₄ aqueous solution (Solution B), small amounts of Solutions A and B were added to adjust the pH to 3.

### Preparation Example 3. pH 5 Solution Preparation

After mixing 520 mL of 0.05 M citric acid aqueous solution (Solution A) and 480 mL of 0.1 M Na₂HPO₄ aqueous solution (Solution B), small amounts of Solutions A and B were added to adjust the pH to 5.

### Preparation Example 4. pH 6 Solution Preparation

After mixing 400 mL of 0.05 M citric acid aqueous solution (Solution A) and 600 mL of 0.1 M Na₂HPO₄ aqueous solution (Solution B), small amounts of Solutions A and B were added to adjust the pH to 6.

### Preparation Example 5. pH 7 Solution Preparation

After mixing 649 mL of 0.1 M KH₂PO₄ aqueous solution (Solution A) and 351 mL of 0.1 M NaOH aqueous solution (Solution B), the pH was adjusted to 7 by adding small amounts of Solution A and I.

### Preparation Example 6. pH 8 Solution Preparation

After mixing 521 mL of 0.1 M KH₂PO₄ aqueous solution (Solution A) and 479 mL of 0.1 M NaOH aqueous solution (Solution B), the pH was adjusted to 8 by adding small amounts of Solution A and I.

### Preparation Example 9. Preparation of a Solution of pH 9

After mixing 568 mL of 0.2 M boric acid aqueous solution (Solution A) and 345 mL of 0.1 M NaOH aqueous solution (Solution B), the pH was adjusted to 9 by adding solutions A and B in small amounts.

### Preparation Example 7. Preparation of a Solution of pH 10

After mixing 470 mL of 0.2 M boric acid aqueous solution (Solution A) and 530 mL of 0.1 M NaOH aqueous solution (Solution B), the pH was adjusted to 10 by adding solutions A and B in small amounts.

### Preparation Example 8. Preparation of a Solution of pH 12

To 100 mL of 0.1 M NaOH aqueous solution (Solution A), 500 mL of 0.1 M KCl aqueous solution (Solution B) was added, the volume was increased to 1 liter with purified water, and the pH was adjusted to 12 by adding solutions A and B in small amounts.

### Experimental Example 1. Solubility by pH

50 mL of pH 1 to 12 solution and tertiary purified water were taken precisely and placed in a 50 mL clear glass vial, and an excess amount (50 mg) of KR-67607 was weighed and added and stirred for 12 hours. Then, it was allowed to stand for 20 minutes to settle the undissolved excess of KR-67607, and the supernatant was taken and filtered through a 0.2 μm PVDF filter as a stock solution.

The standard was prepared by accurately weighing 15 mg of KR-67607, placing it in a 50 mL volumetric flask, using diluent (0.02 M potassium dihydrogen phosphate aqueous solution:acetonitrile=50:50 (v/v)) to completely dissolve/mark KR-67607, and filtering the liquid through a 0.45 μm PVDF filter as the standard.

The HPLC analysis conditions were as follows
Detector: ultraviolet-visible absorption spectrophotometer (measuring wavelength: 254 nm)
Column: 250×4.6 mm, 5μm, C18 packing column
Column temperature: 25±3° C.
Injection volume: 25 μL
Flow rate: 1 mL/min
Mobile Phase: Gradient

TABLE 1

| | Mobile Phase A | Mobile Phase B |
|---|---|---|
| Min | (0.02M KH₂PO₄) | (Acetonitrile) |
| 0 | 70 | 30 |
| 10 | 70 | 30 |
| 20 | 60 | 40 |
| 40 | 60 | 40 |
| 50 | 50 | 50 |
| 100 | 50 | 50 |

The results are shown in FIG. 2 and Table 2.

TABLE 2

| | Solubility (ug/ml) |
|---|---|
| pH 1 | 47.83 |
| pH 3 | 46.08 |
| pH 5 | 42.52 |
| pH 6 | 43.61 |
| pH 7 | 43.77 |
| pH 8 | 41.95 |
| pH 9 | 45.97 |
| pH 10 | 44.53 |
| pH 12 | 55.32 |
| Purified water | 18.83 |

As can be seen in FIG. 2 and Table 2, it was confirmed that there was almost no difference in solubility according to pH for KR-67607 in both purified water and pH 1 to 12 solutions.

### Experimental Example 2. Solubility as a Function of HP-β-CD Concentration, Without pH Adjustment (Purified Water)

Various concentrations of HP-β-CD aqueous solutions were prepared by adding appropriate amounts of HP-β-CD at 0.5%, 1%, 2%, 3%, 4%, and 5% (w/v) in tertiary purified water. An excess of KR-67607 was added to the above HP-β-CD solution and stirred for 24 hours, then the supernatant was collected, filtered through a 0.2 μm PVDF filter, and 1 mL was added to a 10 mL volumetric flask and labeled with methanol.

The standard was 50 mg of KR-67607 accurately weighed and placed in a 50 mL volumetric flask and completely dissolved/stained with methanol. 1, 2, 4, 6, 8, 10 mL of the standard was accurately taken and placed in a 20 mL volumetric flask, and the one labeled with methanol was used as the standard.

The UV analysis conditions are as follows
Wavelength: 255 nm
Cell: 10 mm Quartz cell

KR-67607 concentration (μg/mL)=(UV absorbance of the sample−y-intercept of the standard curve)/(slope of the standard curve)×dilution factor of the sample. [Formula]

TABLE 3

| HP-β-CD | the solubility of |
|---|---|
| concentration (%, w/v) | KR-67607 (mg/ml) |
| 0.5 | 1.20 |
| 1.0 | 2.55 |
| 2.0 | 4.23 |
| 3.0 | 6.00 |
| 4.0 | 6.91 |
| 5.0 | 7.25 |

As shown in FIG. 3 and Table 3, the solubility of KR-67607 in water increased with increasing HP-β-CD concentration.

### Experimental Example 3. Solubility by HP-β-CD Concentration at pH Adjustment

Various concentrations of HP-β-CD aqueous solutions were prepared by adding 1%, 2%, 3%, and 4% (w/v) of HP-β-CD to tertiary purified water (pH 5.5), pH 7.4 phosphate buffer, and 0.04 M NaOH aqueous solution (pH 12.5). An excess of KR-67607 was added to the above HP-β-CD solution and stirred for 24 hours, then the supernatant was collected and filtered through a 0.2 μm PVDF filter, and 1 mL was added to a 10 mL volumetric flask and labeled with methanol.

For the standard solution, 50 mg of KR-67607 was accurately weighed and placed in a 50 mL volumetric flask and completely dissolved/stained with methanol. 1, 2, 4, 6, 8, 10 mL of the standard solution was accurately taken and placed in a 20 mL volumetric flask, and the one labeled with methanol was used as the standard.

TABLE 4

| | | | 0.04M NaOH |
|---|---|---|---|
| HP-β-CD | purified | phosphate | aqueous |
| concentration | water | buffer | solution |
| (%, w/v) | (pH 5.5) | (pH 7.4) | (pH 12.5) |
| 1.0 | 2.55 | 2.59 | 3.16 |
| 2.0 | 4.23 | 4.29 | 5.72 |
| 3.0 | 6.00 | 6.00 | 7.90 |
| 4.0 | 6.91 | 6.79 | 9.61 |

As shown in FIG. 4 and Table 4, the entrapment capacity of HP-β-CD to KR-67607 was confirmed to be basic >acidic, neutral.

### Experimental Example 4. Appearance

HP-β-CD aqueous solutions of various pH were prepared by adding 3% (w/v) appropriate amount of HP-β-CD to 0.08M NaOH aqueous solution, tertiary purified water, and 1M HCl aqueous solution. To each of the above different HP-β-CD solutions, 0.15% (w/v) KR-67607 was added and stirred for 1 hour, and then potassium dihydrogen phosphate, sodium chloride, and povidone K90 were mixed to a concentration of 0.68%, 0.435%, and 1.2% (w/v), respectively, to dissolve completely. The pH of the solution was adjusted to around 7.4 with 0.1 N HCl or 0.1 N NaOH aqueous solution, and then the total volume was adjusted by adding sterile purified water. The prepared solution was filtered through a 0.2 μm PVDF filter and evaluated for comparative appearance against a black background, and the results are shown in FIG. 5.

As can be seen in FIG. 5, the entrapment ability of HP-β-CD for KR-67607 was enhanced when the solution before entrapment was under alkaline conditions.

### Examples 1 to 30. Preparation of Pharmaceutical Compositions Comprising Cyclodextrin or Cyclodextrin Derivatives

Pharmaceutical compositions comprising cyclodextrins or cyclodextrin derivatives were prepared according to the ingredients and contents of Table 5 to Table 7 below. The contents in Table 5 indicate mg/mL of each component in the pharmaceutical composition.

Specifically, a basic solution was prepared by adding a base to a glass beaker and stirring with a magnetic stirrer at room temperature. To the obtained basic solution, cyclodextrin or cyclodextrin derivative was added and mixed. KR-67607 was then added and mixed and dissolved by stirring with a magnetic stirrer at room temperature, and then buffer, isotonic agent, viscosity regulator (thickener), pH regulator, etc. were added and stirred with a magnetic stirrer at room temperature to prepare KR-67607 eye drop pharmaceutical composition.

TABLE 5

| | Example | | | | | | | | | |
|---|---|---|---|---|---|---|---|---|---|---|
| | 1 | 2 | 3 | 4 | 5 | 6 | 7 | 8 | 9 | 10 |
| KR-67607 | 0.1 | 0.75 | 0.75 | 1.5 | 1.5 | 1.5 | 3 | 3 | 5 | 10 |
| 2-hydroxypropyl-β- | 2 | 15 | 30 | 15 | 30 | 60 | 50 | 80 | 100 | 220 |
| cyclodextrin | | | | | | | | | | |
| sodium hydroxide | 1.6 | 1.6 | 1.6 | 1.6 | 1.6 | 1.6 | 1.6 | 1.6 | 1.6 | 1.6 |
| sodium chloride | 5 | 4.8 | 4.35 | 4.35 | 4.35 | 3.7 | 3.65 | 3.2 | 2.65 | — |
| Povidone K90 | 12 | 12 | 12 | 12 | 12 | 12 | 12 | 12 | 12 | 12 |
| Potassium dihydrogen | 6.8 | 6.8 | 6.8 | 6.8 | 6.8 | 6.8 | 6.8 | 6.8 | 6.8 | 6.8 |
| phosphate | | | | | | | | | | |
| Sterile Purified Water | Residual | Residual | Residual | Residual | Residual | Residual | Residual | Residual | Residual | Residual |
| | Amount | Amount | Amount | Amount | Amount | Amount | Amount | Amount | Amount | Amount |

TABLE 6

| | Example | | | | | | | | | |
|---|---|---|---|---|---|---|---|---|---|---|
| | 11 | 12 | 13 | 14 | 15 | 16 | 17 | 18 | 19 | 20 |
| KR-67607 | 1.5 | 1.5 | 1.5 | 1.5 | 1.5 | 1.5 | 1.5 | 1.5 | 1.5 | 1.5 |
| α-Cyclodextrin | 30 | | | | | | | | | |
| β-Cyclodextrin | | 30 | | | | | 60 | | | |
| γ-Cyclodextrin | | | 30 | | | | | | | |
| Sulfobutyl ether-β- | | | | 30 | | | | 60 | | |
| cyclodextrin | | | | | | | | | | |
| 2-Hydroxyethyl-β- | | | | | 30 | | | | 60 | |
| cyclodextrin | | | | | | | | | | |
| 2-Hydroxypropyl-γ- | | | | | | 30 | | | | 60 |
| cyclodextrin | | | | | | | | | | |
| sodium hydroxide | 1.6 | 1.6 | 1.6 | 1.6 | 1.6 | 1.6 | 1.6 | 1.6 | 1.6 | 1.6 |
| sodium chloride | 4 | 4.1 | 4.1 | 4.5 | 4.2 | 4.35 | 3 | 4.15 | 3.65 | 3.8 |
| Povidone K90 | 12 | 12 | 12 | 12 | 12 | 12 | 12 | 12 | 12 | 12 |
| Potassium dihydrogen | 6.8 | 6.8 | 6.8 | 6.8 | 6.8 | 6.8 | 6.8 | 6.8 | 6.8 | 6.8 |
| phosphate | | | | | | | | | | |
| Sterile Purified Water | Residual | Residual | Residual | Residual | Residual | Residual | Residual | Residual | Residual | Residual |
| | Amount | Amount | Amount | Amount | Amount | Amount | Amount | Amount | Amount | Amount |

TABLE 7

| | Example | | | | | | | | | |
|---|---|---|---|---|---|---|---|---|---|---|
| | 21 | 22 | 23 | 24 | 25 | 26 | 27 | 28 | 29 | 30 |
| KR-67607 | 1.5 | 1.5 | 1.5 | 1.5 | 1.5 | 1.5 | 1.5 | 1.5 | 1.5 | 1.5 |
| 2-Hydroxypropyl-β- | 30 | 30 | 30 | 30 | 30 | 30 | 30 | 30 | 30 | 30 |
| cyclodextrin | | | | | | | | | | |
| Sodium Hydroxide | | 1.6 | 1.6 | 1.6 | 1.6 | 1.6 | 1.6 | 1.6 | 1.6 | 1.6 |
| Potassium Hydroxide | 1.7 | | | | | | | | | |
| Sodium Chloride | 4.85 | | | | | 4.35 | 4.35 | 4.35 | 4.35 | 4.35 |
| Glycerin | | | 6.9 | | | | | | | |
| Mannitol | | | | 13.5 | | | | | | |
| PEG400 | | | | | 30 | | | | | |
| Povidone K90 | 12 | 12 | 12 | 12 | 12 | | | 12 | 12 | 12 |
| Hypromellose | | | | | | 6 | | | | |
| (HPMC 2910) | | | | | | | | | | |
| Carbomer | | | | | | | 0.5 | | | |
| Potassium dihydrogen | 6.8 | 6.8 | 6.8 | 6.8 | 6.8 | 6.8 | 6.8 | | | |
| phosphate | | | | | | | | | | |
| Boric Acid | | | | | | | | 3 | | |
| Sodium dihydrogen | | | | | | | | | 6 | |
| phosphate | | | | | | | | | | |
| Citric Acid Anhydrous | | | | | | | | | | 15 |
| Sterile Purified Water | Residual | Residual | Residual | Residual | Residual | Residual | Residual | Residual | Residual | Residual |
| | Amount | Amount | Amount | Amount | Amount | Amount | Amount | Amount | Amount | Amount |

### Example 31. Ex vivo Corneal Penetration and Evaluation of Intraocular Tissue Distribution after in vivo Instillation

NTX-101_vC (Example 5): DP with HP-beta CD as solubilizer

NTX-101_VT: DP with D-a-Tocopherol polyethylene glycol 1000 succinate (TPGS) as solubilizer

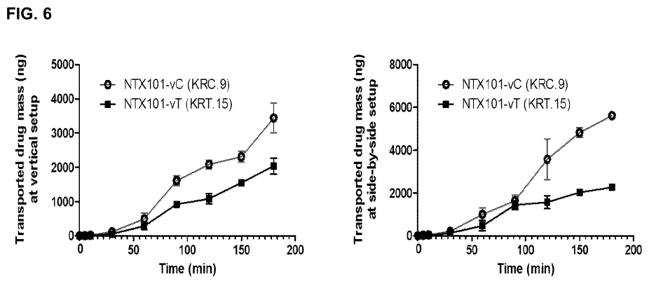

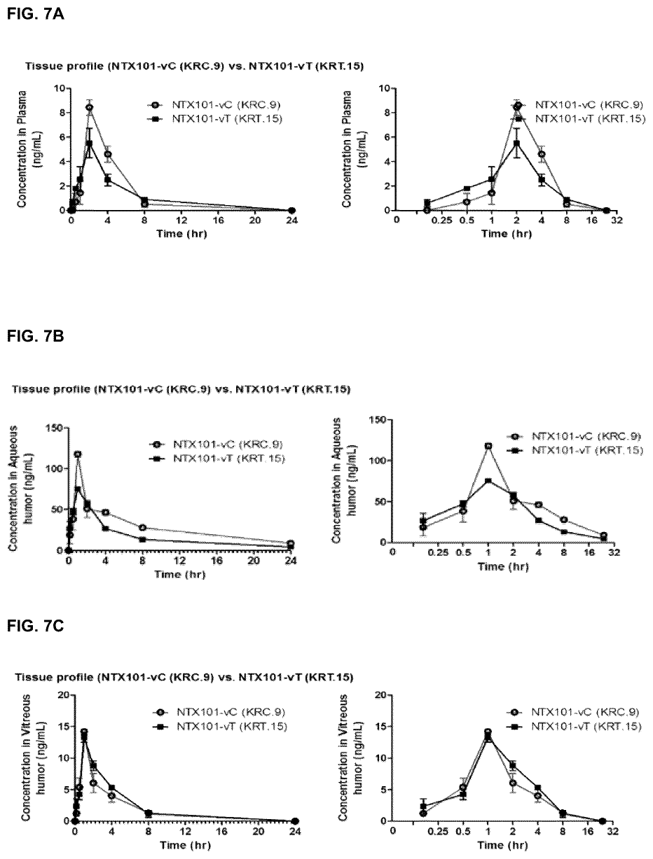

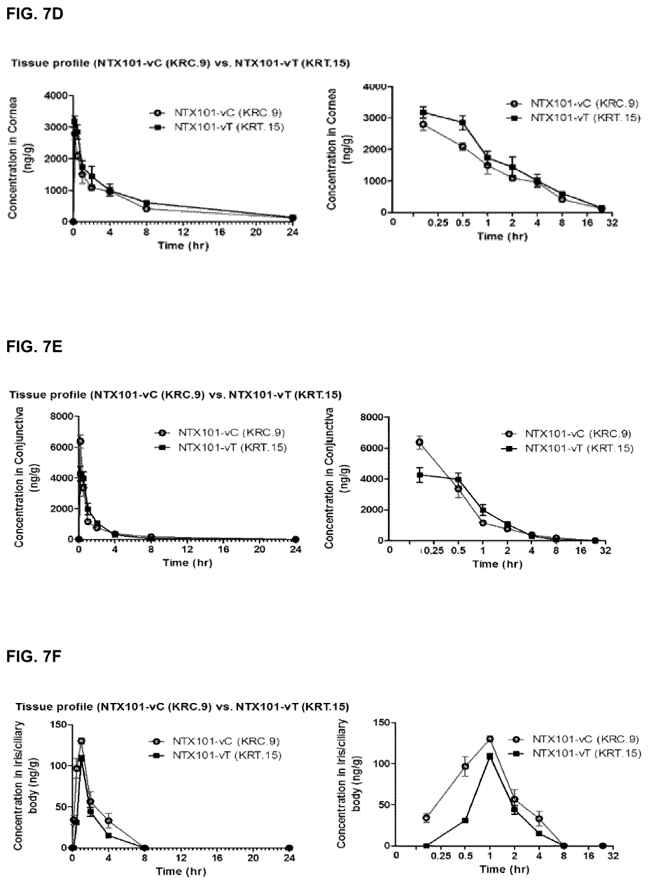

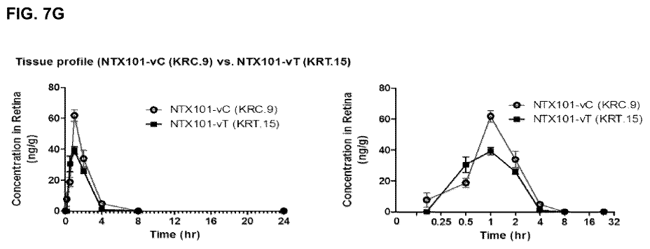

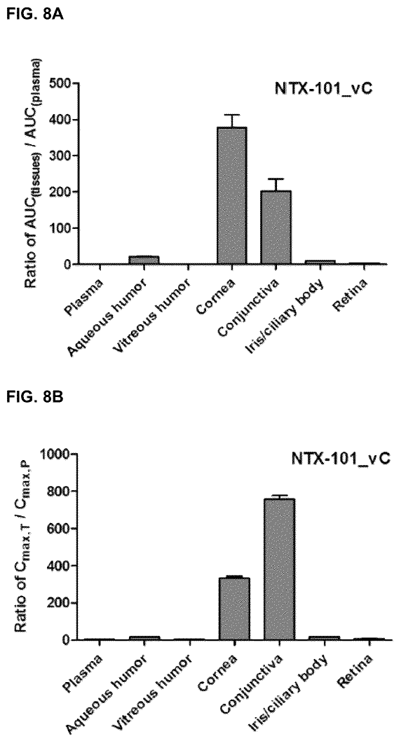

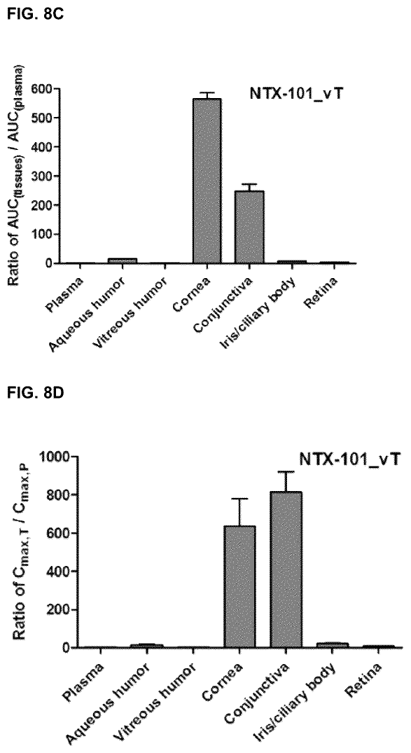

The study analyzed the permeation through the rabbit comea and intraocular distribution after instillation of NTX-101 ophthalmic solution for the two formulations (NTX-101_vC and NTX-101_VT) (FIGS. 6 to 8). Pharmacokinetic analysis was performed to determine the absorption, distribution, and disappearance of the test formulation components in the blood and ocular tissues over time after administration of the formulation. The corneal permeability and in vivo pharmacokinetic profiles were compared to determine how similar the two formulations were.

Prior to analyzing biopsy or post corneal permeation samples from rabbits in the test group treated with the test formulation, the LC/MS method for the qualitative analysis of the samples was validated to ensure that the samples were quantified under consistent analytical conditions. Standards were prepared by diluting the NTX-101 standard by concentration and analyzed by LC/MS, and the extraction efficiency (or recovery) of the drug product in the formulation was verified by comparing the LC/MS analysis results of standards prepared by diluting the two test formulations with HBSS buffer or donated plasma and ocular tissue extracts (blank biomatrices) under the same conditions. By quantifying these standards, good linearity calibration curves were obtained for buffer, plasma, and each ocular tissue, which were used to calculate the concentration of extracted drug in ex vivo corneal permeability test samples and in the plasma and ocular tissues of test rabbits.

To validate the LC/MS method, an intra-day validation (intra-day precision) was performed using the standard solution in plasma and an inter-day validation (inter-day precision) performed with different batches of the standard solution for 5 days, and the CV values were found to be within 80-120% (confirming the precision of the method). Using carbamazepine as an internal standard (IS), which can be measured under the same analytical conditions as the loteprednol etabonate standard, we confirmed that the extraction efficiency was consistent when repeating the extraction process of the components in the biosample. An extraction method with a recovery rate of about 90% was established, and it was also verified that the theoretical concentration value and the concentration value calculated through the calibration line were 80˜120% for different batches of the standard extraction sample, showing almost the same accuracy.

Based on these validated extraction methods and instrumental analysis methods, biological samples from rabbits treated with both test products were analyzed and relevant pharmacokinetic parameters were calculated by PK program, and the concentration of the drug in the blood reached the peak blood concentration after 2 hours.

In ocular structures, peak concentrations were reached in the cornea and conjunctiva at 10 minutes and in the aqueous humor, vitreous humor, retina, and iris/ciliary body at 1 hour. Both formulations showed the same trend for time to maximum concentration (Tmax). In general, the coefficient of variation (CV, %) of the calculated values of Cmax, half-life (t1/2), AUC up to 24 hours or AUC up to infinite time was within 20% for both formulations. The bioequivalence test method based on noncompartmental analysis (NCA) of WinNonlin program was used to statistically evaluate the equivalence between the two formulations. As a result, a t-test was performed on the difference between the mean values of each pharmacokinetic parameter of the two formulations, and it was found that there was no statistically significant difference (P-value>0.05) in Cmax and AUC in plasma and vitreous humor. However, the pharmacokinetics of the two formulations were not equivalent in aqueous humor, retina, and iris/ciliary body, with peak concentrations in the cornea and AUC values in the conjunctiva showing no significant differences at the 90% confidence interval. In the in vivo rabbit ocular model, NTX-101_vC was 1.56, 1.49, 1.19, and 1.57 times higher than NTX-101_VT in the anterior chamber, conjunctiva, iris/macula, and retina, respectively. In the ex vivo rabbit corneal permeability test, NTX-101_vC showed approximately 2-fold higher mass transport rate and permeability values than NTX-101_VT in both vertical and side-by-side experimental setups, indicating that ex vivo experiments are helpful in predicting the pharmacokinetics of this formulation during in vivo instillation.

### Example 32. Efficacy Evaluation of KR-67607 in an Ocular Hypertensive Glaucoma Mouse Model

Support This research was supported by a grant of the Korea Health Technology R&D Project through the Korea Health Industry Development Institute (KHIDI), funded by the Ministry of Health & Welfare, Republic of Korea (grant number : HI15C1142)

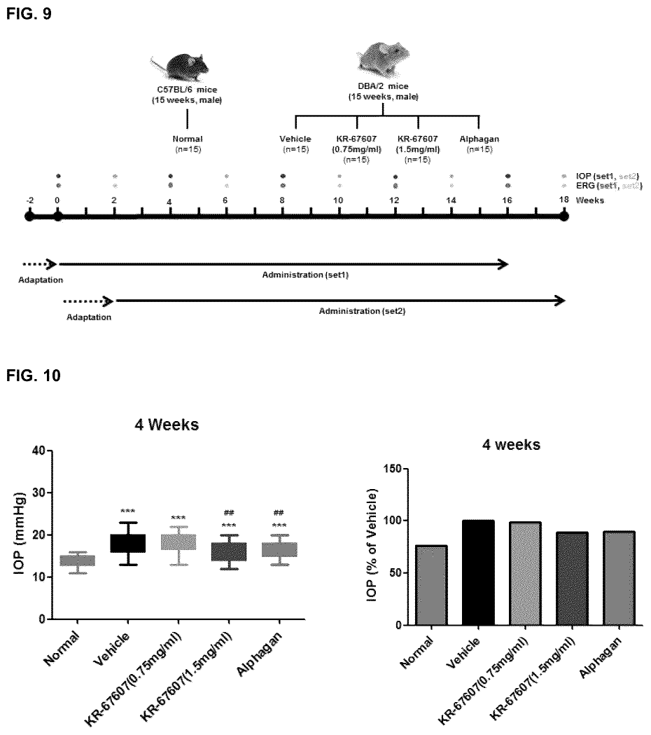

In this study, we evaluated the efficacy of KR-67607 in a mouse model of glaucoma using DBA/2 mice, which are congenitally predisposed to persistently elevated intraocular pressure (FIG. 9).

Vehicle, test substance (KR-67607 0.75 mg/ml and KR-67607 1.5 mg/ml), and positive control (Alphagan P (brimonidine tartrate ophthalmic solution) 0.15%, marketed by Elergan, Korea) were instilled in both eyes at 20 μl twice daily for 16 weeks after the acclimatization period, and compared by measuring IOP and ERG at 4-week intervals.

IOP was measured after the acclimatization period and then every 4 weeks. IOP was measured in respiratory anesthetized mice according to the manufacturer's instructions and the mean value presented after six measurements was recorded. Tonometry was re-measured if the standard deviation displayed by the tonometer itself was greater than 3.5.

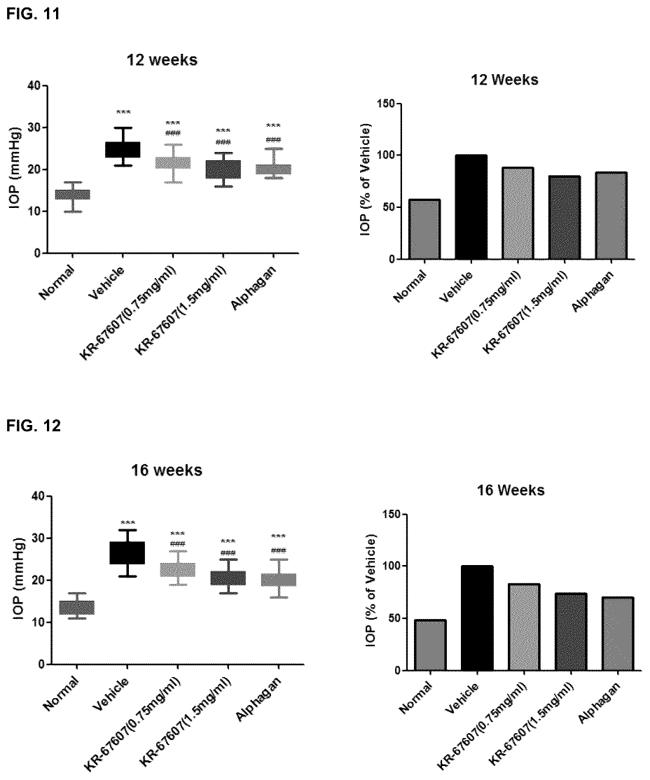

After the acclimatization period, the IOPs of the mice in each group were measured at 4-week intervals for 16 weeks. When comparing the IOP of DBA/2 mice, a glaucoma model, to the control group, there was no significant difference at week 0 of treatment at the age of 4 months. Then, at 5 months of age, IOP began to show a significant difference at week 4 of treatment (FIG. 10) and continued to increase until week 16 of treatment (FIGS. 11 and 12). There were no significant IOP differences between the control, drug, and vehicle groups before drug administration. Significant IOP reductions were seen in the high dose KR-67607 and Alphagan groups compared to the control group starting at week 4 (FIG. 10). IOP-lowering effects were observed in the low-dose KR-67607 group starting at week 12 (FIGS. 11 and 12). By week 16, IOP was reduced by 26% and 30% in the high-dose KR-67607 and Alphagan groups, respectively, compared to the significantly increased IOP in the control group (FIG. 12). IOP in the low concentration KR-67607 group was also reduced by 16%.

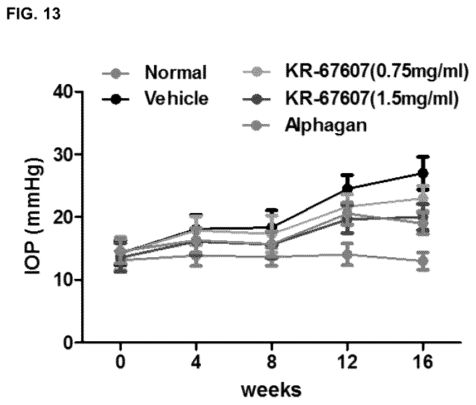

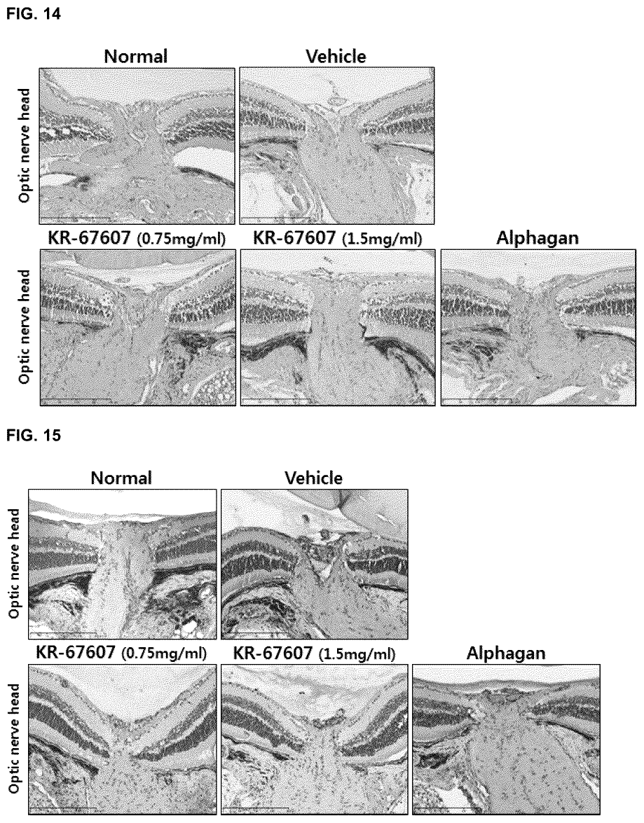

IOP measurements showed that IOP increased with increasing weekly age in DBA/2 mice compared to C57BL/6 mice and was reduced by KR-67607 treatment (FIG. 13). There was no significant difference in IOP-lowering effect between the KR-67607 and Alphagan groups.

In summary, the IOP of DBA/2 mice was elevated for 16 weeks, and the elevated IOP was reduced by KR-67607 treatment. The IOP-lowering effect of KR-67607 was seen at both the 0.75 mg/ml and 1.5 mg/ml concentrations, but the 1.5 mg/ml concentration resulted in lower IOP. The IOP-lowering effect of 1.5mg/ml KR-67607 was similar to the positive control, Alphagan. Based on the IOP measurements, KR-67607 appears to have an IOP-lowering effect.

Retinal electrophoresis was performed according to the manufacturer's instructions after mice were anesthetized by instilling 20 μl of paracaine eye drops 0.5% in both eyes. The electrodes were placed on the scalp, tail, and cornea, respectively, and a certain amount of monochromatic white light stimulation was applied to perform retinal electroretinography. The response value of the stimulated retina and the amplitude from the crest of the a-wave to the peak of the b-wave were measured and evaluated as an indicator of retinal function.

By analyzing the b-wave, which shows the function of bipolar cells in the retina, retinal electroretinograms can infer retinal dysfunction and optic nerve damage. To determine the effect of KR-67607 on retinal dysfunction and optic nerve damage, the electroretinograms of mice in the study were measured at 4-week intervals for 16 weeks. The electroretinograms were quantified by analyzing responses obtained by exposing the left and right eyes of the mice to −0.9, 0.3, and 1.2 log cd×s/m² of light, respectively. Compared to normal C57BL/6 mice, DBA/2 mice showed a significant decrease in retinal electroretinograms as early as week 4 of treatment at the age of 5 months, which was followed by a significant decrease in the left eye only at week 16 of treatment at the age of 8 months. Before drug administration, there was no significant difference in retinal electroretinograms between the control, drug, and positive control groups. No significant change was seen at week 4. After 8 weeks of treatment, there was a significant increase in retinal electroretinograms in the Alphagan group compared to the control group at 0.3 and 1.2 log cd×s/m² stimulation in the right eye, and a significant increase in retinal electroretinograms in the right eye only at 0.3 log cd×s/m² stimulation in the high-dose KR-67607 group. This effect was only transient at week 8, and no significant difference was seen from week 12.

In summary, the retinal electroretinograms of DBA/2 mice showed a significant decrease compared to the normal group (p<0.001), but a significant decrease was not seen in some eyes at week 16. At week 8, only KR-67607 and Alphagan treatment groups showed a significant increase compared to the vehicle group, but the effect was not sustained, so the effect of the drugs on retinal dysfunction could not be confirmed by retinal electroretinograms.

After drug administration was terminated, the mice's eyes were enucleated and tissues were analyzed (FIGS. 14 to 26).

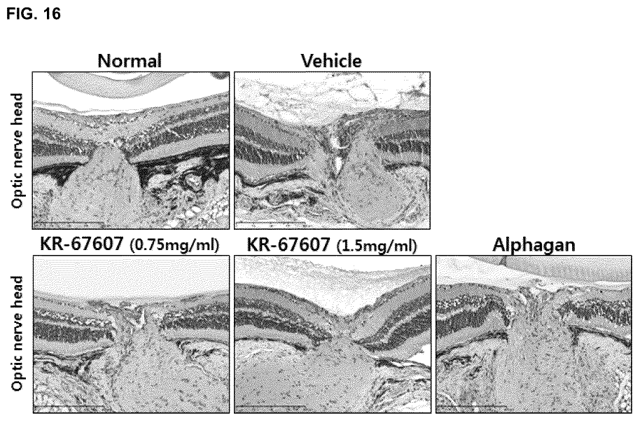

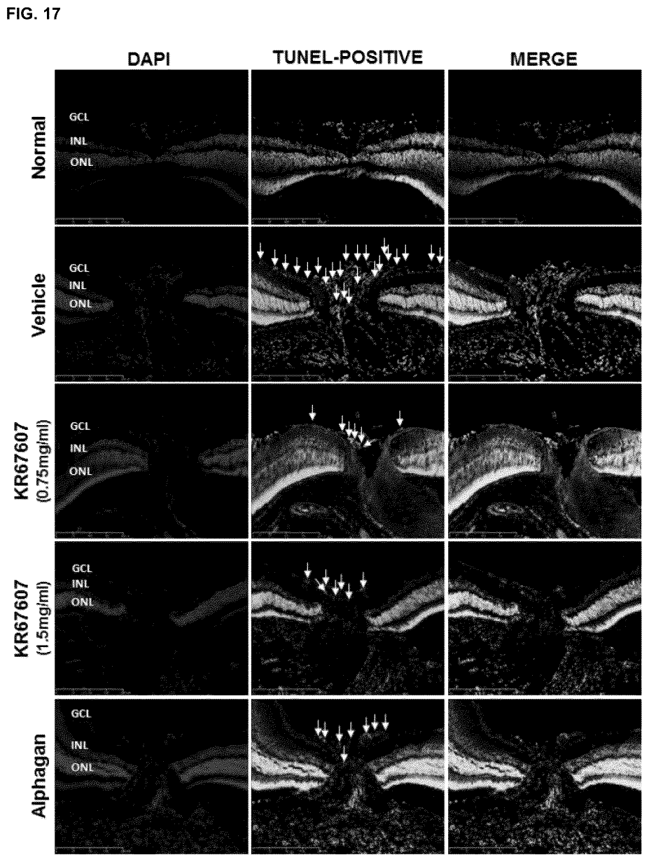

Progressive degeneration of the structure of the optic nerve head is seen in glaucoma as a result of optic nerve damage. The optic nerve papilla area was analyzed by H&E staining, Caspase-3, 9, and TUNEL staining in retinal tissue removed and fixed from eyes of mice after 16 weeks of drug treatment. H&E staining showed that DBA/2 mice had advanced cupping or excavation of the optic nerve papilla and thinning of the nerve fiber layer compared to the normal group. These glaucomatous histologic features were alleviated in the KR-67607 and Alphagan groups (FIG. 14). To determine the degree of cell death in the damaged optic nerve papilla tissue, Caspase-3, 9 staining (FIGS. 15 and 16), an indicator of early apoptosis, and TUNEL staining (FIG. 17), an indicator of late apoptosis, were performed. As a result, a large number of cells expressing caspase-3, 9 and TUNEL-positive cells were observed in the depressed optic nerve papillae of the control group, but the number was significantly reduced in the KR-67607 and Alphagan groups.

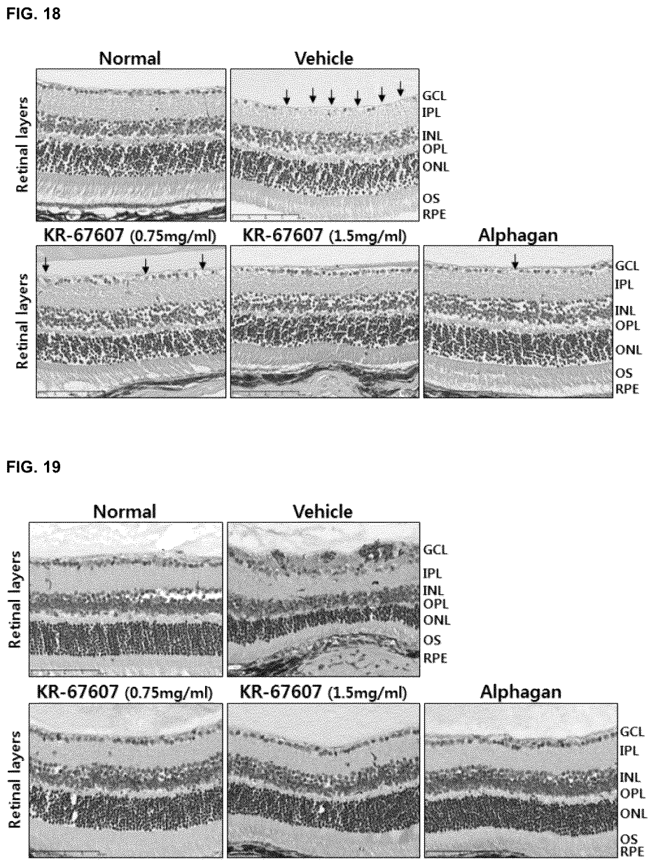

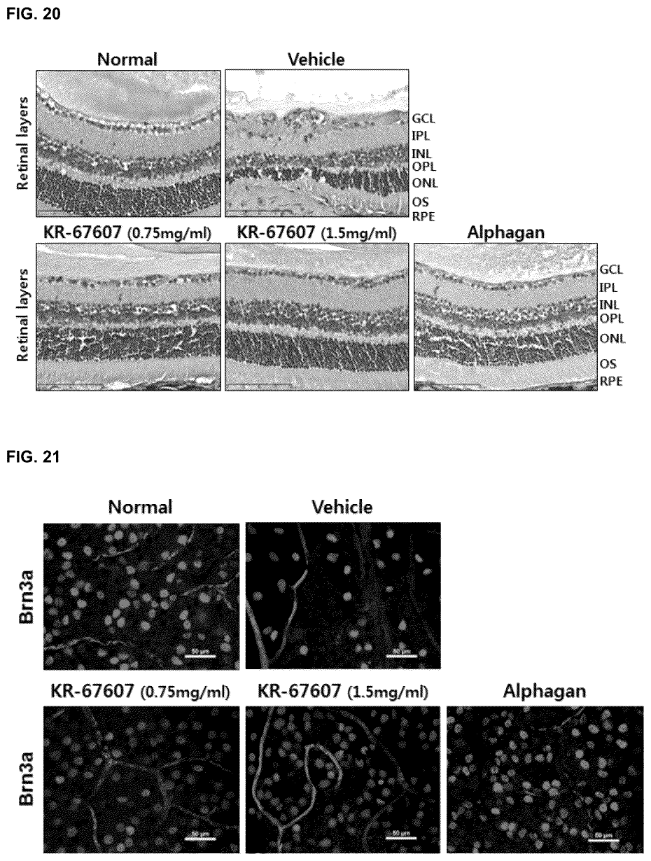

The pathogenesis of glaucoma is known to be caused by the loss of retinal ganglion cells (RGCs) in the gaglion cell layer (GCL) and optic nerve damage. Therefore, we examined the loss of retinal ganglion cells in the GCL layer by H&E staining. It was observed that DBA/2 mice had a large number of retinal ganglion cells dislodged from the retinal ganglion cell layer compared to the normal group (FIG. 18). Among the retinal ganglion cells remaining in the retinal ganglion cell layer, more cells positive for caspase-3 and 9 were also observed in the control group (FIGS. 19 and 20). On the other hand, there was less loss of retinal ganglion cells in the KR-67607 and Alphagan groups (FIG. 18), and the degree of retinal ganglion cell death was relatively reduced (FIGS. 19 and 20).

Brn3a (brain-specific homeobox/POU domain protein 3) is a transcription factor that accumulates in retinal ganglion cells in the rodent retina, and it is known that the expression of Brn3a is reduced by damage to these cells. We confirmed the damage to retinal ganglion cells through changes in Brn3a expression in flat-mounted retinal tissue (FIG. 21). As a result, it was observed that the expression of Bm3a was reduced in DBA/2 mice compared to the normal group, but the expression was relatively increased in the KR-67607 and Alphagan groups. This confirmed that KR-67607 attenuated the damage of glaucomatous retinal ganglion cells.

### Changes in Key Optic Nerve Cells

In the previous test results, we identified changes in retinal ganglion cells caused by KR-67607 in retinal function and optic nerve damage in the DBA/2 mouse model. In order to determine the effects of KR-67607 on retinal ganglion cells and other types of major optic nerve cells in the reduction of retinal function, we analyzed the expression of factors known to be indicators of optic nerve cells in retinal tissues.

### 1. Astrocyte Activation

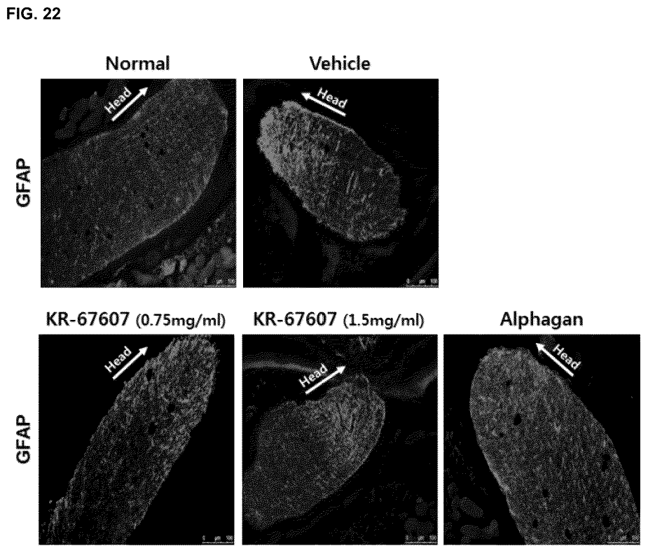

GFAP (glial fibrillary acidic protein) is known to be an indicator of astrocyte and Müller cell activation that occurs in response to retinal stress (Chen H, Weber A J. Expression of glial fibrillary acidic protein and glutamine synthetase by Müller cells after optic nerve injury and intravitreal application of brain-derived neurotrophic factor. Glia. 2002;38(2):115-25). To analyze the changes in astrocytes after optic nerve injury, the optic nerve sections were fluorescently stained with GFAP and the tissues were analyzed (FIG. 22). Although quantitative differences could not be identified through histological immunostaining of GFAP, it was observed that activated astrocytes and Müller cells were concentrated in the optic nerve papilla area with nerve injury in DBA/2 mice. In contrast, GFAP expression was less concentrated in the KR-67607 group, and GFAP expression was more evenly distributed throughout the optic nerve in the normal and Alphagan groups. This suggests that the nerve damage caused by activated astrocytes was significantly reduced in the KR-67607 and Alphagan groups compared to the control group.

### 2. Changes in Retinal Neuron, Cone Photoreceptor and Cone Bipolar Cell

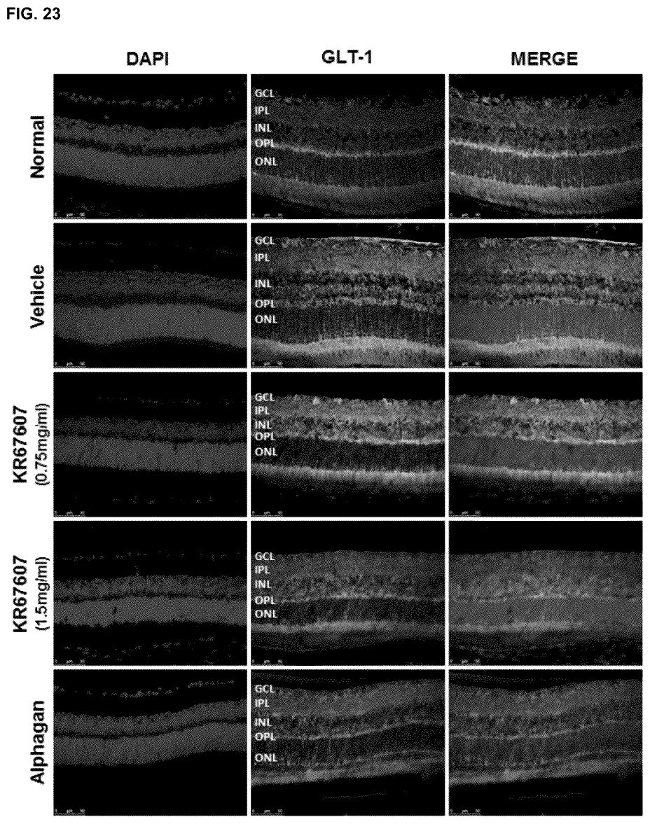

GLT-1 (glutamate transporter 1) is a transporter involved in the transportation of glutamate, a major excitatory neurotransmitter, and is expressed in retinal neurons, cone photoreceptors, and cone bipolar cells. Its expression is known to be increased in glaucoma, especially in closed-angle glaucoma such as DBA/2 mice. GLT-1 expression was increased in the IPL, INL, OPL, and ONL of DBA/2 mice with glaucomatous changes compared to normal mice, but there was no quantitative or qualitative difference between KR-67607 and Alphagan groups (FIG. 23).

### 3. Changes in Amacrine Cells

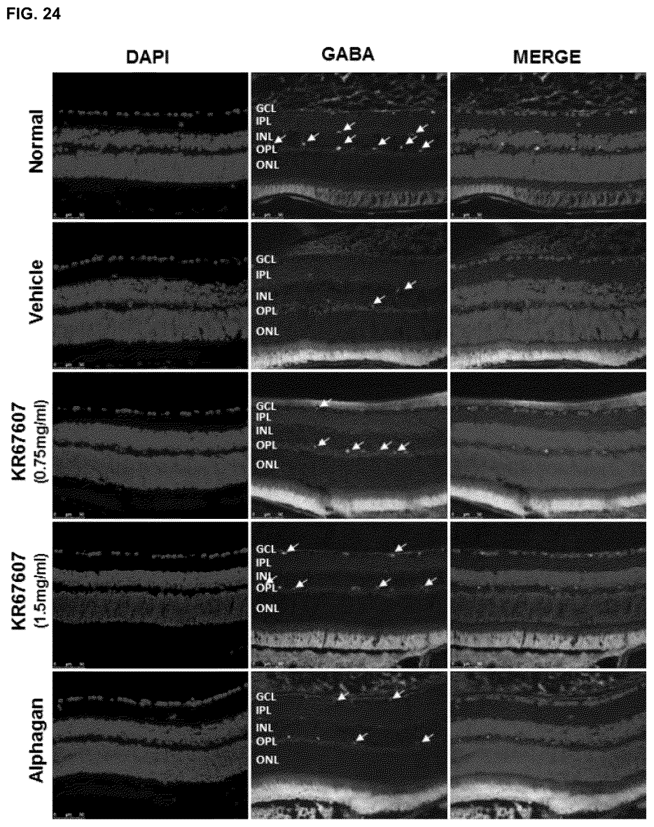

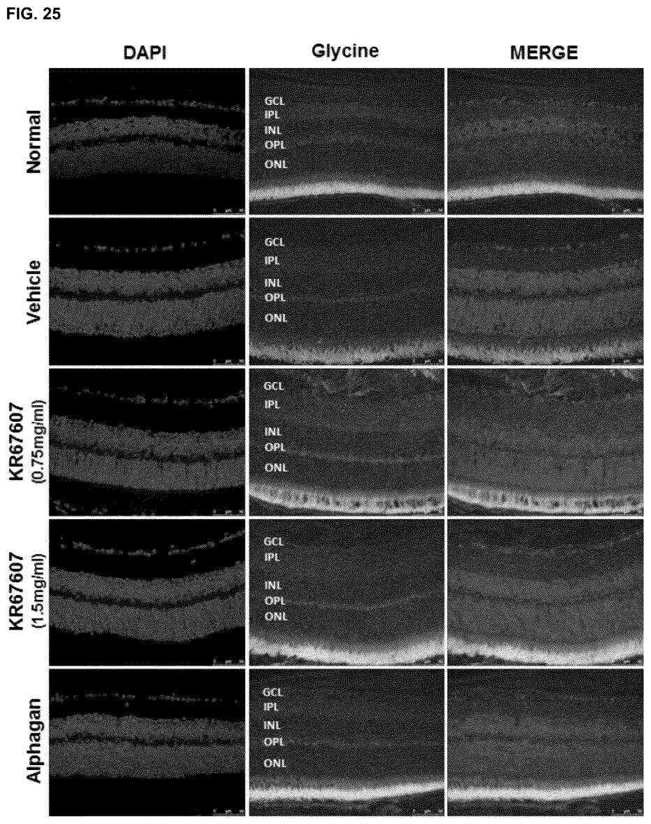

Amacrine cells are inhibitory neurons that release the inhibitory transmitters gamma-aminobutyric acid (GABA) and glycine, which act as intermediaries to detect changes in visual stimuli and make neural contacts. Their expression is known to be abnormal in glaucoma models. GABAergic amacrine cells were observed to be located at the borders of the GCL, INL, and IPL in the normal retina, but their number was reduced in the retina of DBA/2 mice. The reduced GABAergic amacrine cells were relatively increased in the KR-67607-treated and Alphagan-treated groups (FIG. 24). However, for glycinergic amacrine cells, there was no significant difference between all groups (FIG. 25).

### 4. Changes in Microglia

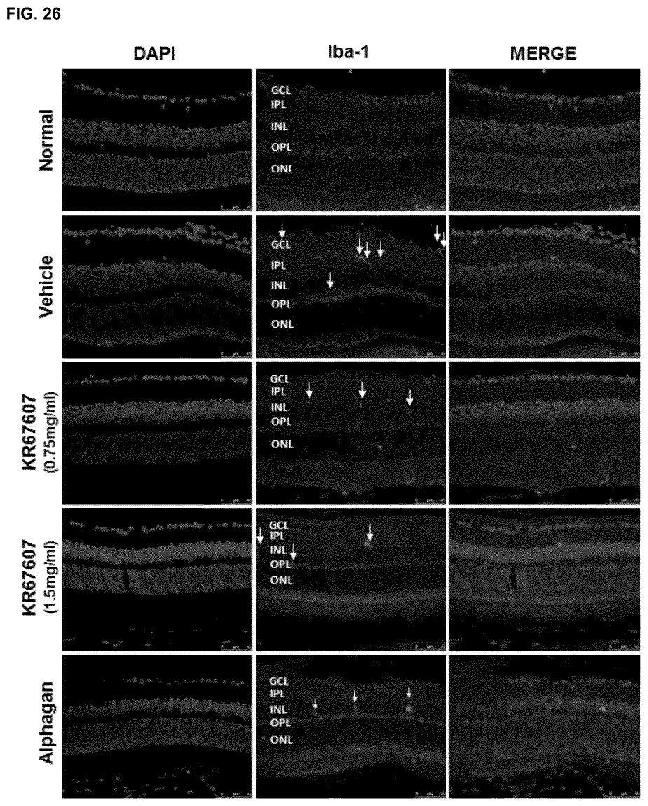

Glaucomatous pathologic tissue changes are accompanied by an immune-inflammatory response, which involves microglia, a type of glial cell. The role of activated microglia in optic nerve damage is also known to be important. Proinflammatory cytokines produced by activated microglia damage retinal ganglion cells and nerves. In DBA/2 mice, the number of microglia positive for Iba-1 (ionized calcium-binding adapter molecule 1), a marker of microglia, was increased compared to the control group, but decreased in the KR-67607 and Alphagan groups (FIG. 26).

In short, H&E staining analysis showed that the damage to the glaucomatous optic nerve papilla observed in the control group was relatively reduced in the KR-67607 group. The number of cells positive for caspase-3, 9, and TUNEL, which indicate the extent of cell death in damaged tissue, was also reduced in the KR-67607 group. In addition, unlike the control group, in which the retinal ganglion cells, the main optic nerve cells of the retina, were significantly reduced in the retinal ganglion cell layer, the degree of cell loss was significantly reduced in the KR-67607 group. In addition, even among the remaining cells in the retinal ganglion cell layer, the percentage of cells undergoing apoptosis was relatively small, as confirmed by staining for early cell death markers Caspase-3 and 9. Furthermore, expression of Bm3a in flat-mounted retinal tissue was increased in the KR-67607 group compared to the control group, where expression was reduced due to damage to retinal ganglion cells. These histologic findings suggest that KR-67607 protects the function of the optic nerve by inhibiting damage to optic nerve tissue, reducing retinal ganglion cell damage and cell death, and attenuating cell exodus. In addition to retinal ganglion cells, other optic nerve cells that perform retinal functions were also analyzed. In the retinal tissue of DBA/2 mice, GFAP-expressing activated astrocytes were concentrated in the optic nerve papilla, causing neuronal damage, whereas in the KR-67607 group, the distribution of activated astrocytes became more even, similar to the control group, reducing optic nerve tissue damage. In the case of GABAergic amacrine cells, which function as inhibitory neurons, the number decreased dramatically in DBA/2 mice, but in the KR-67607 group, the number increased relatively and became a function of the retina. In addition, the number of activated microglia, which was increased in glaucomatous retinal tissue, was reduced in the KR-67607 group, suggesting that the neuronal damage caused by activated microglia was alleviated in the drug group.

In conclusion, ocular administration of KR-67607 lowered the elevated IOP in the glaucoma mouse model and attenuated tissue damage and cell death caused by elevated IOP. It also reduced the damage and loss of retinal ganglion cells, preserved the function of the optic nerve by reducing the reduction of GABAergic amacrine cells, and protected retinal ganglion cells and nerve tissue from neuronal damage caused by neurotoxins and inflammatory substances secreted by them by reducing the number of activated astrocytes and microglia. These results demonstrate that KR-67607 has potent activity against glaucoma caused by elevated intraocular pressure.

### Example 33. IOP-Lowering Efficacy Evaluation Test in Rabbit (New Zealand White) Hypertensive Animal Model

Inhibition of 11β-HSD1 activity in the monkey model confirmed excellent optic nerve protection, i.e., pharmacological efficacy in terms of IOP lowering and optic nerve protection.

KR-67607 Ophthalmic Solution: 1 mL of KR-67607 ophthalmic excipient contains 1.5 mg of KR-67607

Control 1 (Xalatan): Latanoprost 0.05 mg/mL

Control 2 (Timoptic): Timolol maleate 6.83 mg/mL

Control 3 (Trusopt): Dorzolamide hydrochloride 22.26 mg/mL

Control 4 (Xalacom): Latanoprost 50 ug/mL, timolol 5 mg/mL

This study was conducted in male New Zealand White rabbit ocular hypertension model animals (intraocular pressure ≥17 mmHg) induced by scleral vein ligation to determine the effectiveness of KR-67607 eye drops in reducing ocular hypertension after repeated twice daily ophthalmic administration for 11 days.

Sterile physiological saline (negative control), Xalatan, Timoptic, Trusopt, Xalacom, and KR-67607 eye drops were administered twice daily for 11 days, and mortality and general symptoms were monitored, body weight was measured, feed intake was measured, and intraocular pressure was measured. The results of the study were summarized as follows:

No dose-related changes in general condition, body weight and gain, or feed intake were observed in any of the treatment groups.

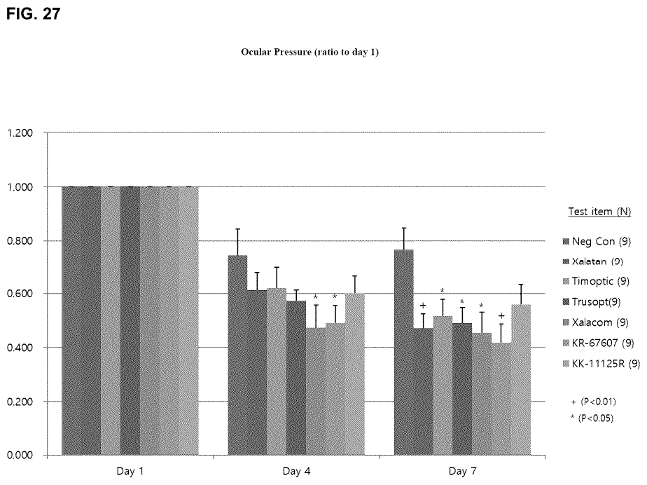

When comparing post-dose IOP from pre-dose to post-dose, greater IOP reductions were observed in all positive control and KR-67607 treatment groups than were observed in the negative control group (FIG. 27).

Specifically, negative control IOPs were reduced by 74.7% and 76.7% on days 5 and 8, respectively, when compared to 100% of pre-dose IOP. In the Xalatan, Timoptic, Trusopt, and Xalacom treatment groups, IOPs were observed to be 61.2%, 62,1%, 57.3%, and 48.1% at day 5 and 47.1%, 51.8%, 49.1%, and 45.9% at day 8, respectively, while IOPs in the KR-67607 treatment group were observed to be 49.0% and 60.1% at day 5 and 41.8% and 56.6% at day 8, respectively.

IOP reductions in the Xalatan, Timoptic, Trusopt, and Xalacom groups were greater than in the negative control group at 18.1%, 16.9%, 23.3%, and 36.7% at Day 5 and 38.6%, 32.5%, 35.9%, and 40.9% at Day 8, respectively, with greater reductions at Day 8 than at Day 5. In the KR-67607 group, the reduction was greater than in the negative control group at 34.4% on day 5 and 45.5% on day 8.

In summary, among the positive controls, the positive control with the lowest IOP based on pre-dose IOP (100%) was the Xalacom treatment group with an observed IOP of 45.4% on day 8, while the KR-67607 treatment group had an observed IOP of 41.7%, a 3.6% greater reduction than the Xalacom treatment group.

### Example 34. Ocular Tissue Harvesting and Analysis

Following Example 33, in order to separate the sample (eyeball) to observe the glaucoma treatment effect on the drug in addition to the change in intraocular pressure caused by the drug administration, the eyeball was enucleated and about 0.2 mL of the aqueous humor was collected, and then the retina (including some choroid) was collected. The collected aqueous humor and retina were placed in 1.5 mL polypropylene tubes pre-marked with sample information, frozen in a deep freezer (approximately −80° C.) until analysis, and delivered to the test site for analysis.

RNA or proteins were extracted from the eyes and analyzed by RT-PCR and western blotting to compare the expression of genes and proteins that can confirm the glaucoma treatment effect or optic nerve protection effect.

In the case of protein expression, the samples collected were contaminated with blood, so accurate results could not be confirmed.

RNA was extracted from the isolated retinal tissue and analyzed by RT-PCR to compare the expression of genes that can confirm the glaucoma treatment effect or retinal protection effect. The tissues were lysed using TrizolTM Reagent (Invitrogen, CA, US) to extract the soluble RNA, and the RNA was precipitated by adding isopropanol (100%; Sigma-aldrich, MO, US).

Subsequently, cDNA was synthesized from the isolated RNA using a cDNA synthesis kit (AccuPower RT Premix; Bioneer, KR) and used for RT-PCR. The primer information used for RT-PCR analysis is as follows.

GFAP_F:
(SEQ ID NO: 1)
GACATCGAGATCGCCACCTA;
GFAP_R:
(SEQ ID NO: 2)
ACGGTCTTCACCACGATGTTC;
TNFα_F:
(SEQ ID NO: 3)
CTCAGATCAGCTTCTCGGGC;
TNFα_R:
(SEQ ID NO: 4)
GTGAGTGAGGAGCACGTAGG;
BAX_F:
(SEQ ID NO: 5)
ACCAAGCTGGTACTCAAGGC;
BAX_R:
(SEQ ID NO: 6
CAAGATGGTCAGCGTTTGCC;
18S rRNA_F:
(SEQ ID NO: 7)
GTAACCCGTTGAACCCCATT;
18S rRNA_R:
(SEQ ID NO: 8)
CCATCCAATCGGTAGTAGCG.

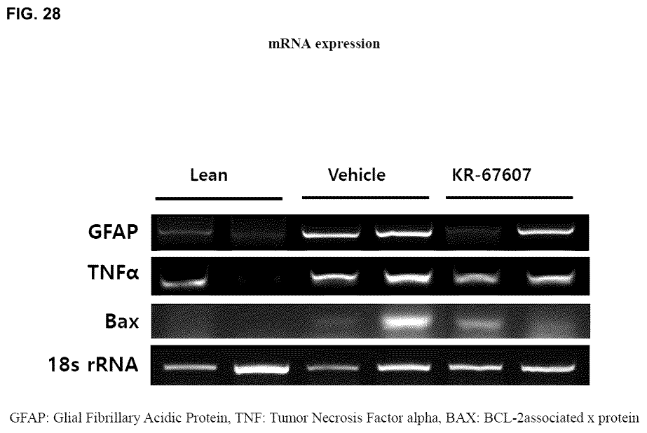

The synthesized DNA strands were then analyzed after separation on a 1.5% agarose gel using gene-specific primers (FIG. 28).

As shown in FIG. 28, the mRNA expression levels of Glial Fibrillary Acidic Protein (GFAP), elevated by high intraocular pressure and glaucoma, a pro-inflammatory response factor, Tumor Necrosis Factor alpha (TNFa), and BCL-2 associated x protein (BAX), a major factor inducing apoptosis, were compared. The results showed that the mRNA expression levels of GFAP, TNFa and BAX were elevated in retinal tissues induced with high intraocular pressure, while the expression levels tended to decrease in retinal tissues of animals treated with KR-67607 eye drops.

### Example 35. Effect of KR-67607 in Cynomolgus Macaque Model of Glaucoma

The purpose of the current study was to observe the effect of KR-67607 on intraocular pressure, color fundoscopy and retinal nerve fiber layer thickness in a cynomolgus macaque model of glaucoma.

Three weeks before the first laser trabeculoplasty, IOP was measured via a tomometer (SPHPR-320-61), once per week for three weeks. The first laser trabeculoplasty was performed in one eye for each macaque. Two weeks later, laser trabeculoplasty was repeated in the same eye (Week 0).

One week after the second laser trabeculoplasty (Week 1), IOP was measured and retinal nerve fiber layer (RNFL) thickness was measured via optical coherence tomography (OCT; RS3000 Advance, Nidek Co. Ltd., Gamagoori, Japan) (SPHPR- 320-58). The day after group assignment, the eyes were instilled with either vehicle, KR-67607 or brimonidine twice a day. On Day 1, the effect of treatment on IOP over time was measured before treatment and 1, 2, 4, and 8 hrs. after instillation. At the same time, blood samples were obtained. From the effects of dosing observed on Day 1, it was determined that 2 hrs. was a reasonable time time-to-test following instillation for subsequent observations.

Intraocular pressures were obtained at Weeks 2, 3, 4, 5, 7 and 9. Optical coherence tomography and color fundus photography (CFP) were performed at Weeks 1, 3, 5, 7, and 9 (SPHPR-320-56).

Following measurements at Week 9, G3 macaques was randomized into two groups based on IOP, and the effect of KR-67607 (1.5 mg/mL, 3.0 mg/mL) on IOP was evaluated.

| Group | Treatment | Concentration | N (eyes) |
|---|---|---|---|
| G1 | Vehicle | — | 6 |
| G2 | Brimonidine tartrate | 0.1% | 6 |
| G3 | KR-67607 | 1.5 mg/mL | 6 |

### RESULTS

### 1. Intraocular Pressure (IOP) Trough Values

In the vehicle-treated group (G1), beginning 1 week following the laser photocoagulation treatment, the mean (+SEM) IOP in the glaucomatous eye was elevated (67.7±3.8 mmHg), compared to the normal, contralateral eye (17.4±1.3 mmHg; Table 1, FIG. 1). Significantly increased IOP was observed for the duration of the study period in the vehicle-treated group. Repeated treatment of glaucomatous eyes with brimonidine (G2) and KR-67607 (G3) for the duration of the study period did not significantly affect trough IOP value.

### 2. Eight Hour IOP Time Course

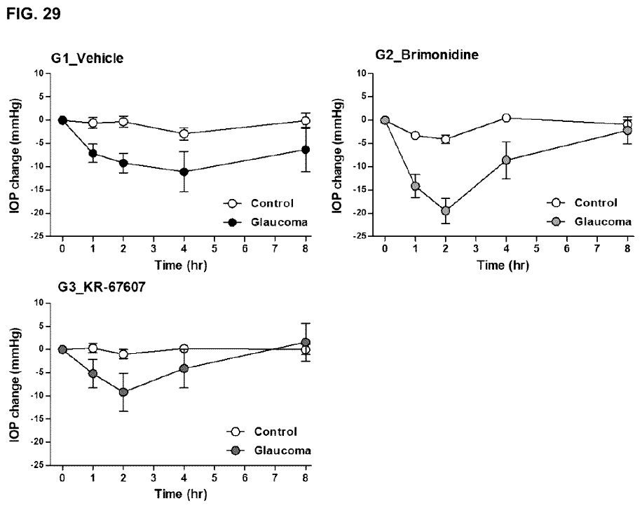

In G1, IOP was decreased 1 hour after instillation and did not return to baseline (“pre”) value 8 hours after dosing (Day 1 dosing, FIG. 29). Brimonidine lowered IOP of the glaucomatous eye, with a maximum decrease of 19.5±2.7 mmHg 2 hours after instillation. Brimonidine also decreased IOP of the contralateral, normal eye with a maximum decrease of 4.1±0.9 mmHg 2 hours after dosing. Intraocular pressure of the contralateral, normal eye returned to baseline 8 hours after instillation. KR-67607 also decreased IOP with a maximum decrease of 9.2±4.1 mmHg at 2 hours after instillation. Unlike brimonidine, KR-67607 did not decrease IOP of the contralateral, normal eye.

### 3. Effects of Test Compounds on IOP Over Weeks

In G1, IOP was either increased (2.6 mmHg) or decreased (−0.5 to −7.9 mmHg) 2hours after instillation. Brimonidine lowered IOP of the glaucomatous eye over the course of the study at every time point. Mean decreases were between 10.6±1.8 and 18.1±1.4 mmHg 2 hours after instillation. Brimonidine also decreased IOP of the normal eye, with a maximum decrease of 4.9±0.8 mmHg. KR-67607 also decreased IOP with a maximum decrease of 10.4±2.2 mmHg 2 hours after instillation in the glaucomatous eye.

However, unlike brimonidine, KR-67607 did not decrease IOP of the contralateral, normal eye.

4. Effects of KR-67607 (1.5, 3.0 mg/mL) on IOP

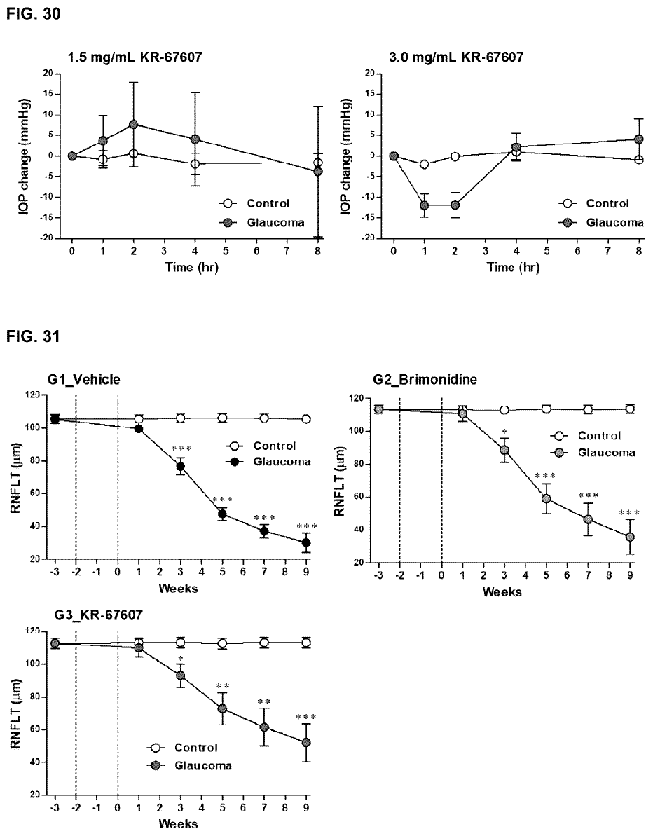

KR-67607 (1.5 mg/mL) did not affect IPO. KR-67607 (3.0 mg/mL) decreased IOP with a maximum decrease of 12.0±2.8 mmHg 1 hour after instillation (Extra dosing; FIG. 30).

### 5. Effects of Test Compounds on Retinal Nerve Fiber Layer (RNFL) Thickness

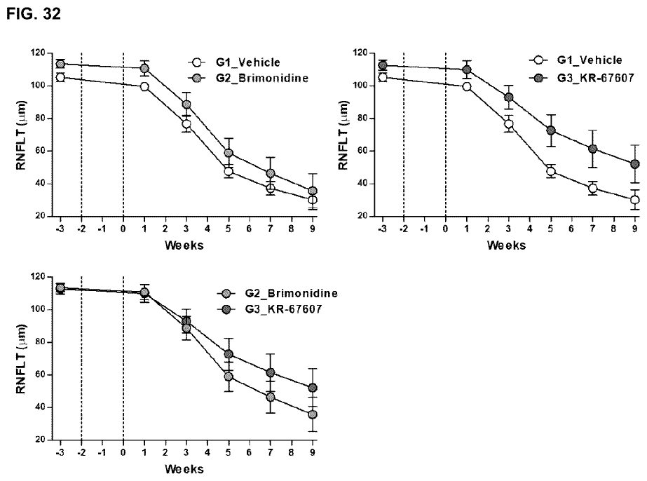

Mean RNFL thickness (total average) in the vehicle-treated group (G1) was significantly decreased three weeks following the second laser treatment, compared to the contralateral, normal eye, which persisted for the duration of the study period (FIGS. 31 and 32) . Repeated treatment of glaucomatous eyes with brimonidine (G2) appeared to inhibit the progression of RNFL thinning. In the KR-67607-treated glaucoma eyes (G3),

RNFL thinning was significantly inhibited compared to vehicle-treated (G1) glaucomatous eyes (See Sections 23.17 and 23.18 for representative RNFL images from G1 and G2). The number of eyes with RNFL thickness greater than 50% compared to baseline at Week 9 in G1, G2, and G3 were 0, 1, 3, respectively.

### 6. Funduscopy

In G1, compared to the contralateral, normal eye, marked optic disc enlargement (cupping) was observed in the glaucomatous eye in five of six animals (except for K-725) from Week 3 to Week 9. In G2, three animals (K-668, K-736, K-771) displayed a lesser degree of cupping compared to that of G1 at Week 3 to Week 5.

At Week 9, one animal (K-771) displayed a small degree of cupping. In G3, four of six animals (except for K-667, K-676) had small optic discs compared to G1 in the glaucomatous eyes at Week 5. At Week 9, three animals (K-669, K-684, K-743) displayed a lesser degree of cupping compared to that of G1 (data not shown).

### SUMMARY

The purpose of the current study was to investigate the effects of KR-67607 on experimental glaucoma in macaques. KR-67607 appeared to decrease IOP in the glaucomatous eyes, although the effect was smaller than that of brimonidine. Both KR-67607 and brimonidine tended to inhibit RNFL thinning, but the inhibitory effect of KR-67607 appeared to be greater than that of brimonidine. The progression of RNFL thinning was slower in eyes with small optic disc.

1. An ophthalmic formulation in the form of an eye drop comprising an inclusion complex of a poorly water-soluble drug of Formula 1 below entrapped in cyclodextrin or a cyclodextrin derivative in an aqueous solution of pH 10 or higher, allowing it to pass through the corneal epithelium and be delivered into ocular tissues: 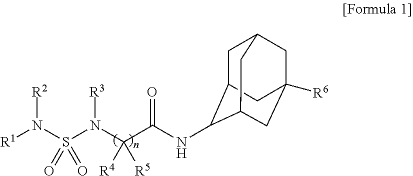
wherein R¹ represents H; C₁-C₆ alkyl; cyano C₁-C₆ alkyl, C₃-C₈ cycloalkyl; benzyl unsubstitutued or substitutued with halogen, C₁-C₆ alkyl or OCX₃ (X is halogen);
phenylethyl; C₁-C₆ alkoxycarbonyl; phenylacetyl; naphthyl; or 5-10 membered aryl substitutued with halogen, C₁-C₆ alkyl, C₃-C₈ cycloalkyl, C₁-C₆ alkoxy, CX₃(X is halogen), OCX₃ (X is halogen), cyano, nitro, or 5-10 membered aryl or heteroaryl;
R² and R³ independently represent C₁-C₆ alkyl; or C₂-C₆ alkenyl; with the proviso that R² and R³ bind together to form a ring structure,
R⁴ and R⁵ independently represent H; or C₁-C₆ alkyl;
R⁶ represents H; OH; COOR⁷; or CONR⁷R⁷;
R⁷ represents H; or C₁-C₆ alkyl; and
n represents an integer of 1-3.

2. The ophthalmic formulation of claim 1, wherein a sufficient concentration of the poorly soluble drug of Formula 1 can be delivered into the ocular tissue via the inclusion complex.

3. The ophthalmic formulation of claim 1, wherein the inclusion complex allows a sufficient amount of the poorly soluble drug of Formula 1 to reach the ocular tissues to prevent cell death due to ischemic injury.

4. The ophthalmic formulation of claim 1, wherein the cyclodextrin derivative is 2-hydroxypropyl-β-cyclodextrin.

5. The ophthalmic formulation of claim 1, wherein the poorly soluble drug of Formula 1 is a compound represented by Formula 1 or a pharmaceutically acceptable salt thereof.

6. The ophthalmic formulation of claim 1, which is provided by a solubilization method comprising the following steps:
a step of preparing a solution in which a solubilization promoter is dissolved in water to obtain a solution having a pH of at least 10.0;
a solubilizer dissolution step in which the solution is mixed with cyclodextrin or a cyclodextrin derivative as a solubilizer;
a compound dissolving step comprising mixing in the solution a compound represented by Formula 1 or a pharmaceutically acceptable salt thereof; and
a pH adjusting step of mixing a pH adjusting agent into the solution.

7. The ophthalmic formulation of claim 6, wherein the method further comprises a step of dissolving in the solution at least one selected from the group consisting of buffers, isotonic agents, viscosity modifiers, antioxidants, and chelating agents.

8. The ophthalmic formulation of claim 6, wherein the solubilizing promoter is at least one selected from the group consisting of a basic substance and a buffer.

9. The ophthalmic formulation of claim 6, wherein the step of dissolving the compound is carried out in a weight ratio of 1:10 to 40 of the compound represented by Formula 1 or a pharmaceutically acceptable salt thereof and the solubilizer.

10. The ophthalmic formulation of claim 6, wherein the compound represented by Formula 1, or a pharmaceutically acceptable salt thereof, included in the eye drop formulation prepared by the above method is 0.01 to 1.0 w/v %.

11. The ophthalmic formulation of claim 6, wherein the osmolarity is from 250 to 340 mosmol.

12. The ophthalmic formulation of claim 1, wherein the pH is between 5 and 9.

13. The ophthalmic formulation of claim 1, wherein the poorly water soluble drug of formula 1 is 4-(2-(6-(2,6-dichloro-4-(trifluoromethyl)phenyl)-4-methyl-1, 1-dioxido-1,2,6-thiadiazinan-2-yl)acetamido)adamantane-1-carboxamide or a pharmaceutically acceptable salt thereof.

14. A pharmaceutical composition comprising an inclusion complex, wherein the poorly soluble drug of Formula 1 is entrapped within 2-hydroxypropyl-β-cyclodextrin in an aqueous solution of pH 10 or higher: 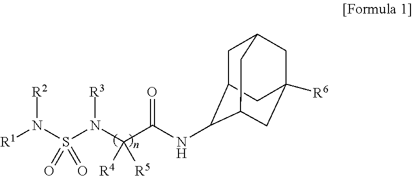
wherein R¹ represents H; C₁-C₆ alkyl; cyano C₁-C₆ alkyl, C₃-C₈ cycloalkyl; benzyl unsubstitutued or substitutued with halogen, C₁-C₆ alkyl or OCX₃ (X is halogen);
phenylethyl; C1-Cs alkoxycarbonyl; phenylacetyl; naphthyl; or 5-10 membered aryl substitutued with halogen, C₁-C₆ alkyl, C₃-C₈ cycloalkyl, C₁-C₆ alkoxy, CX₃(X is halogen), OCX₃ (X is halogen), cyano, nitro, or 5-10 membered aryl or heteroaryl;
R² and R³ independently represent C₁-C₆ alkyl; or C₂-C₆ alkenyl; with the proviso that R² and R³ bind together to form a ring structure,
R⁴ and R⁵ independently represent H; or C₁-C₆ alkyl;
R⁶ represents H; OH; COOR⁷; or CONR⁷R⁷;
R⁷ represents H; or C₁-C₆ alkyl; and
n represents an integer of 1-3.

15. A pharmaceutical composition for administration as an ophthalmic formulation in the form of an eye drop to a patient in need of optic nerve protection, comprising a poorly water soluble drug of Formula 1 below to be delivered into ocular tissues by passing through the corneal epithelium: 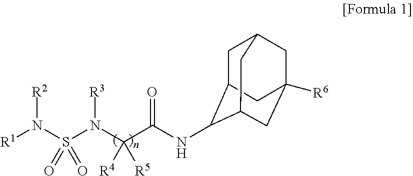
wherein R¹ represents H; C₁-C₆ alkyl; cyano C₁-C₆ alkyl, C₃-C₈ cycloalkyl; benzyl unsubstitutued or substitutued with halogen, C₁-C₆ alkyl or OCX₃ (X is halogen);
phenylethyl; C₁-C₆ alkoxycarbonyl; phenylacetyl; naphthyl; or 5-10 membered aryl substitutued with halogen, C₁-C₆ alkyl, C₃-C₈ cycloalkyl, C₁-C₆ alkoxy, CX₃(X is halogen), OCX₃ (X is halogen), cyano, nitro, or 5-10 membered aryl or heteroaryl;
R² and R³ independently represent C₁-C₆ alkyl; or C₂-C₆ alkenyl; with the proviso that R² and R³ bind together to form a ring structure, R⁴ and R⁵ independently represent H; or C₁-C₆ alkyl;
R⁶ represents H; OH; COOR⁷; or CONR⁷R⁷;
R⁷ represents H; or C₁-C₆ alkyl; and
n represents an integer of 1-3.

16. The pharmaceutical composition of claim 15, which is for the prevention or treatment of ischemic optic neuropathy.

17. The pharmaceutical composition of claim 15, which is for the prevention or treatment of glaucoma.

18. The ophthalmic formulation of claim 1, wherein the poorly water soluble drug of Formula 1 protects the ocular tissue and/or optic nerve by a mechanism of inhibition of intracellular cortisol production and/or intracellular activation of the antioxidant factor Nrf2/HO-1.

19. The pharmaceutical composition of claim 14, wherein the poorly water soluble drug of Formula 1 protects the ocular tissue and/or optic nerve by a mechanism of inhibition of intracellular cortisol production and/or intracellular activation of the antioxidant factor Nrf2/HO-1.

20. The pharmaceutical composition of claim 15, wherein the poorly water soluble drug of Formula 1 protects the ocular tissue and/or optic nerve by a mechanism of inhibition of intracellular cortisol production and/or intracellular activation of the antioxidant factor Nrf2/HO-1.
